(19) United States
(12) Patent Application Publication
(10) Pub. No.: US 2025/0078962 A1
(43) Pub. Date: Mar. 6, 2025

(54) SYSTEMS AND METHODS FOR PRODUCING PERSONAL CARE PRODUCTS WITH IMPROVED ENVIRONMENTAL SUSTAINABILITY

(71) Applicant: ELC MANAGEMENT LLC, Melville, NY (US)

(72) Inventors: Eva Claire Thompson, Astoria, NY (US); Paul Scott, Pelham, NY (US)

(21) Appl. No.: 18/516,663

(22) Filed: Nov. 21, 2023

Related U.S. Application Data
(60) Provisional application No. 63/536,888, filed on Sep. 6, 2023.

(51) Int. Cl. G16C 20/50; G16C 20/10

(52) U.S. Cl. CPC G16C 20/50; G16C 20/10

(57) ABSTRACT

Techniques for producing personal care products with environmentally-preferred ingredients may include, for each of a plurality of candidate components, determining a respective e-hazard adjustment indicative of an amount of environmental hazard posed by waste that is generated by utilizing components of a component category of the candidate component in ingredients of personal care products, and modifying an initial e-impact rating of the candidate component based on the e-hazard adjustment to obtain an enhanced e-impact rating of the candidate component. Techniques may additionally or alternatively include determining respective e-impact ratings for a plurality of component categories of components utilized in ingredients of personal care products. Component categories may be delineated based on component chemical structures, feedstock sources, and production processes. Ingredients may be selected based on comparisons of component e-impact ratings (which may or may not be enhanced), and selected ingredients may be utilized to produce formulations of personal care products.

### CROSS-REFERENCE TO RELATED APPLICATIONS

This application claims the benefit of U.S. Application No. 63/536,888, filed Sep. 6, 2023, and entitled “SYSTEMS AND METHODS FOR PRODUCING PERSONAL CARE PRODUCTS WITH IMPROVED ENVIRONMENTAL SUSTAINABILITY”, which is incorporated herein by reference in its entirety.

### TECHNICAL FIELD

This application generally relates to systems, methods, and techniques for producing personal care products with improved environmental and ecosystem sustainability.

### BACKGROUND

The concept of chemical component and ingredient degradation to environmentally benign byproducts is increasingly important in the personal care products industry due to at least consumer sentiment and global regulatory directions. The personal care products industry has been incorporating various green chemistry concepts and techniques to design chemical products and processes that attempt to reduce the generation of environmentally hazardous substances during the production of personal care products such as cosmetics, skin care products, sun care products, hair care products, personal cleansing products, fragrances, and other products used for personal hygiene, beauty, and wellbeing.

“Green Score” tools have been developed to evaluate and provide a measure of how well chemical components, formulations, ingredients, and the use of such components, formulations, and ingredients in the production of personal care products align with green chemistry principles and support environmental sustainability. Accordingly, a Green Score generated by a Green Score tool for a component utilized in an ingredient of personal care products may be a quantitative indication of the environmental impact of processing the component for use in a personal care product. An enterprise may factor in the Green Scores of various components to develop formulations of ingredients, which may then be utilized in the manufacture and production of personal care end products. Some present-day Green Score tools address four of twelve principles of green chemistry that are widely utilized across a majority of chemical industries, namely the principles of safer chemicals and compounds, increased energy efficiencies, use of renewable feedstocks, and real-time analysis to prevent pollution. As such, these present-day Green Score tools incorporate metrics of endpoints within three associated categories of green chemistry, namely, those of human health (HH), ecosystem health (ECO), and environmental impact (ENV). For example, a Green Score tool which is currently being utilized within the personal care products industry incorporates the metrics of acute toxicity, ocular toxicity, and dermal toxicity endpoints of the human health category, the metrics of bioaccumulation, persistence, and aquatic toxicity endpoints of the ecosystem category, and the metrics of feedstock sourcing and greenhouse gas emissions endpoints of the environmental impact category to generate Green Scores of components which may be utilized in formulations of ingredients of personal care products.

Another endpoint of the environmental impact category of green chemistry relates to waste that is produced during the synthesis and other processes utilized to produce personal care products and ingredients thereof. One commonly utilized metric of waste generation utilized in chemical industries is an “E-factor”, which refers to a measurement of environmental acceptability of a chemical manufacturing process. The E-factor of a chemical manufacturing process may be generally defined using the equation:

E-factor=kilograms (kgs) of waste produced/kilograms of desired product

The amount (kg) of reaction waste generated can be determined by summing the mass of every material used in the reaction or process and subtracting the mass of the end product and materials that are generated by the chemical reaction or process, that are not the end product, and that are not environmentally favorable waste (e.g., any valorized or recycled byproducts, solvents, etc.). However, E-factors are typically calculated for only chemical manufacturing or processing steps and do not take into account the environmental impact of waste generated from other production steps or processes, such as the procuring and the initial processing of starting materials and other reagents, purification processes, etc. Accordingly, E-factors are only able to provide an incomplete view on the holistic waste impact of the overall production of end products. Consequently, the E-factors of only the chemical processes within the production of an end product or an E-factor comparison between two synthetic routes may be meaningless or at the most only marginally useful without the context of the entire amount of waste produced throughout the end product's complete production process, such as from procuring feedstock to producing the final end product (e.g., “cradle-to-gate”). Further, present-day E-factors typically address only mass-based quantification, which is a significant factor in the personal care products industry, and attempts to do so are not standardized, thus further limiting the usefulness of the present-day E-factors as a valid measure of environmental sustainability of personal care products and their production processes.

### SUMMARY

In an embodiment, a method of producing a personal care product with environmentally preferred ingredients includes obtaining a plurality of candidate components (e.g., candidate chemical components) for an ingredient of a personal care product and, for each candidate component included in the plurality of candidate components, determining a respective initial e-impact rating of the each candidate component, where the respective e-impact rating of the each candidate component is indicative of an environmental impact of utilizing the each candidate component in ingredients of personal care products. The method also includes, for each candidate component included in the plurality of candidate components, determining a respective e-hazard adjustment indicative of a respective amount of environmental hazard posed by waste that is generated by utilizing one or more components, of a respective component category in which the each candidate component is included, in the ingredients of personal care products. The respective component category may be included in a plurality of categories of components used or for use in the ingredients of personal care products, where each component category of the plurality of component categories includes a respective plurality of components used or for use in the ingredients of personal care products. The plurality of component categories may be delineated based on chemical structures of the components, feedstock sources of the components, and production processes of the components, for example. Additionally, the method includes, for each candidate component included in the plurality of candidate components, modifying the respective initial e-impact rating of the each candidate component based on the respective e-hazard adjustment corresponding to the respective component category of the each candidate component, thereby generating a respective enhanced e-impact rating of the each candidate component. The method further includes selecting, based on a comparison of the enhanced e-impact ratings of the plurality of candidate components, an ingredient that includes a particular component of the plurality of candidate components, and providing an indication of the selected ingredient to at least one of a computer application or a user interface to thereby cause the selected ingredient to be utilized in the manufacturing of the personal care product.

In an embodiment, a method of producing a personal care product with environmentally preferred ingredients includes obtaining an indication of a plurality of candidate components (e.g., candidate chemical components) for an ingredient of a personal care product and, for each candidate component included in the plurality of candidate components, determining a respective component category, of a plurality of categories of components used in ingredients of personal care products, in which the each candidate component is included. The plurality of component categories may be delineated based on chemical structures of the components, feedstock sources of the components, and production processes of the components, for example, and each component category of the plurality of component categories may include a respective plurality of components used or for use in the ingredients of personal care products. The method may also include, for each candidate component included in the plurality of candidate components, determining a respective e-impact rating of the respective component category, where the respective e-impact rating of the respective component category may be indicative of an environmental impact posed by waste that is generated by utilizing at least some of the respective plurality of components of the respective component category in the ingredients of personal care products. The method may further include, for each candidate component included in the plurality of candidate components, assigning the respective e-impact rating of the respective component category as a respective e-impact rating of the each candidate component. The method may still further include selecting, based on a comparison of the e-impact ratings of the plurality of candidate components, an ingredient that includes a particular candidate component of the plurality of candidate components, and providing an indication of the selected ingredient to at least one of a computer application or a user interface, e.g., to thereby cause the selected ingredient to be utilized in the manufacturing of the personal care product.

### BRIEF DESCRIPTION OF THE DRAWINGS

FIG. 1 depicts a block diagram of an example system for producing personal care products with environmentally preferred ingredients.

FIG. 2 depicts a flow diagram of an example method for producing personal care products with environmentally preferred ingredients, which may be implemented by the system of FIG. 1.

FIG. 3 depicts a flow diagram of an example method producing personal care products with environmentally preferred ingredients, which may be implemented by the system of FIG. 1.

FIG. 4 depicts an example of a component categorization structure or framework which may be generated and/or utilized by embodiments of the systems, methods, and/or techniques described herein.

### DETAILED DESCRIPTION

The accompanying figures, where like reference numerals refer to identical or functionally similar elements throughout the separate views, together with the detailed description below, are incorporated in and form part of the specification, and serve to further illustrate embodiments of concepts that include the claimed embodiments and explain various principles and advantages of those embodiments.

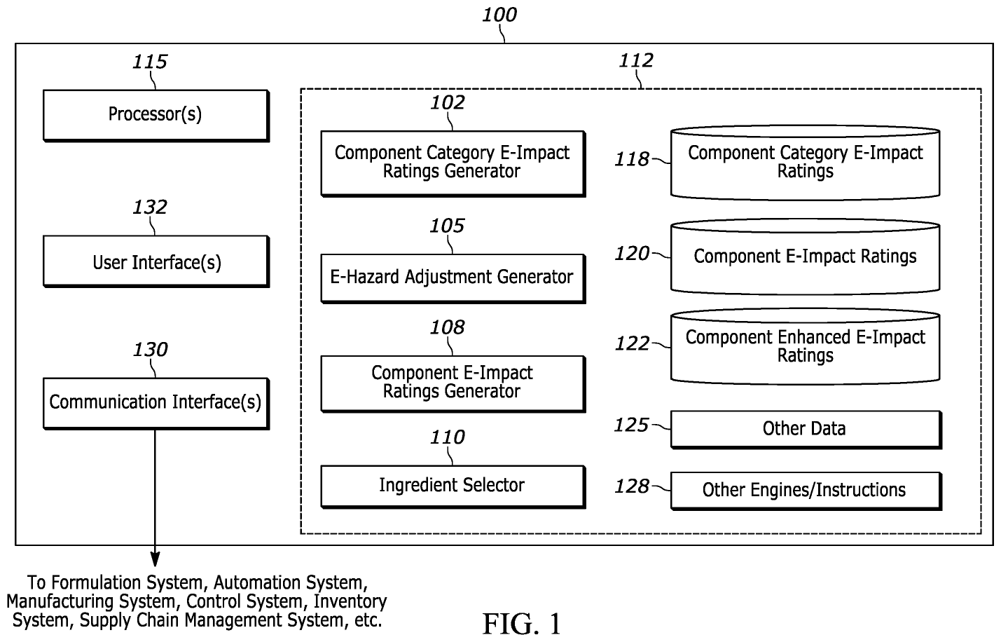

FIG. 1 depicts a block diagram of an example system 100 for producing personal care products using environmentally preferred ingredients. The system 100 may include a combination of computing-related components, parts, or portions, such as hardware, firmware, and software parts, portions, units, modules, and/or other types of computing-related components, as well as various data communication channels for communicating data between and among the various computing-related components, parts, or portions of the system 100. In embodiments, the system 100 may include additional and/or alternate computing-related components, parts, or portions other than those shown in FIG. 1, and/or the system 100 may omit one or more of the computing-related components, parts, or portions shown in FIG. 1. Further, the system 100 may be owned and/or operated by an enterprise or organization, such as a formulator, manufacturer, or other type of provider or producer of personal care products.

The system 100 for producing personal care products using environmentally preferred ingredients may include one or more engines 102-110. For example, as illustrated in FIG. 1, the system 100 includes a component category e-impact ratings generator engine 102, an e-hazard adjustment generator engine 105, a component e-impact ratings generator engine 108, and an ingredient selector engine 110, one or more of which, in embodiments, may be implemented at least in part by a respective set of computer-executable instructions that are stored on one or more memories or data storage devices 112 of the system 100 and that are executable by one or more processors 115 of the system 100. As utilized herein, the prefix “e-” generally refers to “environmental.” Thus, an “e-impact” rating refers to an environmental impact rating, an “e-hazard” adjustment refers to an environmental hazard adjustment, and the like. As further utilized herein, other than the discussion of the computing-related components discussed above, the term “component” generally refers to a chemical component, and particularly when the term “component” is utilized with respect to producing personal care products and/or environmental concepts such as e-factors, e-impact ratings, e-hazards, and the like. As such, the component category e-impact ratings generator engine 102 and the component e-impact ratings generator engine 108 are respectively and interchangeably referred to herein as the chemical component category e-impact ratings generator engine 102 and the chemical component e-impact ratings generator engine 108, a candidate component may be a candidate chemical component, a component category may be a chemical component category, a component e-impact rating may be a chemical component e-impact rating, etc.

The one or more memories 112 may include one or more tangible, non-transitory memories which may be included in, for example, one or more computing devices, a data bank, a data cloud, or any suitable data storage platform. The memories 112 may store various modules or components of the system 100 such as the engines 102, 105, 108, 110 and one or more data stores 118-122. For example, as illustrated in FIG. 1, the one or more memories 112 of the system 100 store a component category e-hazard adjustments data store 118, a component e-impact ratings data store 120, and a component enhanced e-impact ratings data store 122. The one or more memories 112 may additionally store other data 125 and other engines and/or computer-executable instructions 128 which may be utilized by the system 100 to produce personal care products using environmentally preferred ingredients. Generally speaking, at least a portion of the system 100 may include and/or be implemented on one or more servers or computing devices which may be implemented, for example, by a bank of servers, a cloud computing system, one or more networked computing devices, or any other suitable arrangement of one or more computing devices.

The system 100 may include one or more communication interfaces 130 which may communicatively connect the system 100 to one or more communication and/or data networks. Such communication and/or data networks may include one or more public, private, wired, and/or wireless communication networks and/or data networks which can communicatively connect the system 100 with one or more other remote computing devices and/or systems (which may include, in some implementations, one or more personal electronic devices (PEDs) or computers, formulation systems, manufacturing systems, process control systems, automation systems, other types of computing systems related to producing personal care products, etc.). In some embodiments, the one or more communication interfaces 130 may additionally or alternatively communicatively connect the system 100 to one or more local computing devices and/or systems which may include, in some implementations, one or more computers associated with the enterprise or organization of the system 100. The local computing devices may include Personal Electronic Devices (PEDs) operated by agents of the enterprise or organization, such as laptops, tablets, smart phone or devices, in-vehicle computers, etc. In some embodiments, the system 100 may include one or more user interfaces 132, which may include any type of user interface which is locally provided by the system 100, for example, screens, touchscreens, keyboards, touch pads, mice, microphones, speakers, scanners, cameras, and/or other optical, auditory, and/or physical user interface devices.

It should be appreciated that each of the data stores 118-125 may be implemented using one or more databases and/or one or more data storage devices or systems. Although the data stores 118-125 are shown in FIG. 1 as being separate and distinct data stores 118-125, this is merely for purposes of clarity of discussion and is not intended to be limiting. Indeed, any two or more of the data stores 118-125 may at least partially share data platform hardware and/or software resources, if desired. For example, any two or more of the data stores 112-125 may be implemented by a data bank or a data cloud. Further, in some embodiments, two or more of the data stores 112-125 may be implemented at least partially as an integral data store. Still further, in some embodiments (not shown in FIG. 1), at least one of the data stores 112-125 may be located remotely with respect to the system 100 and may be accessible to the system 100 via the communication interface 130 and one or more communication and/or data networks.

Additionally, although the engines 102-110 are shown in FIG. 1 as being separate and distinct engines 102-110, this is merely for the purposes of clarity of discussion and is not meant to be limiting. Indeed, any two or more of the engines 102-110 may be implemented by a single or integral engine and/or by a single or integral set of computer-executable instructions, if desired. For example, although the present document distinguishes between the component e-impact ratings generator engine 108 and the component category e-impact ratings generator engine 102, in some implementations, the component e-impact ratings generator engine 108 and the component category e-impact ratings generator engine 102 may be an integral e-impact ratings generator engine (not shown in FIG. 1). Further, in some embodiments (also not shown in FIG. 1), at least one of the engines 102-110 may be implemented remotely with respect to the system 100 and may be communicatively connected with the system 100 via the communication interface 130 and one or more communication and/or data networks.

The system 100 may operate to produce personal care products using environmentally preferred ingredients by utilizing one or more of the engines 102-110. For example, the component e-impact ratings generator 108 may determine a respective, initial e-impact rating of each component of a plurality of candidate components which may be utilized in generating ingredients of personal care products. Generally speaking, a “component” or “chemical component,” as interchangeably utilized herein, generally refers to feedstock that has been processed (e.g., materially or otherwise initially processed) into a component having a form that is able to be utilized for further processing (which generally, but not necessarily, includes chemical processing of the component and optionally along with other components) into an ingredient of a personal care product. Additionally, as utilized herein and generally speaking, the initial e-impact rating of a component may be indicative of one or more environmental effects, consequences, or impacts resulting from including or utilizing the component in producing an ingredient of a personal care product. In some implementations, the component e-impact ratings generator 108 may discover, determine, or otherwise generate the initial e-impact rating of a candidate component, e.g., in-line with performing one or more other techniques for producing personal care products described herein, and the component e-impact ratings generator 108 may store any discovered or generated initial component e-impact ratings in the component e-impact ratings data store 120. The initial e-impact rating of a component may be determined by using any suitable technique. In an example, for a candidate component, the component e-impact ratings generator 108 may provide input information related to the candidate component to a present-day Green Score tool (which may be included in the system 100, in embodiments), and may obtain the initial component e-impact rating from an output of the present-day Green Score tool. In an example, the component e-impact ratings generator 108 may obtain a candidate component's initial e-impact rating from the component e-impact ratings data store 120, e.g., in-line with performing one or more other techniques for producing personal care products described herein. The component e-impact ratings data store 120 may store respective e-impact ratings of a plurality of different components, where the stored component e-impact ratings have been previously discovered, determined, or otherwise generated by the system 100 and/or by one or more other parties.

In yet another example, for a candidate component, the component e-impact ratings generator 108 may determine the initial e-impact rating of the candidate component to be the e-impact rating of a category of components in which the candidate component is included. Generally, and as referred to herein, categories of components which may be utilized and processed into ingredients of personal care products (also interchangeably referred to herein as “component categories”) may be delineated based on one or more component characteristics which affect or impact environmental sustainability (e.g., either in a positive or negative manner) when such components are processed into ingredients of personal care products. Said another way, a plurality of components may be classified or grouped into respective component categories based on the components' respective environmentally-impacting characteristics. As such, a “component category” is interchangeably referred to herein as a “component classification,” or “component class.” A component category may include multiple sub-categories, a sub-category may include multiple respective sub-categories, and so on. Examples of such component characteristics based on which a plurality of components may be categorized, classified, or grouped may include, for example, raw chemical structures of components (e.g., similarities and/or differences of raw chemical structures of components); various types, locations, and procedures used to procure feedstock sources of components and optionally the similarities and/or differences therebetween; various types and/or numbers of production processes (material, chemical, purification, etc.) that are applied to components, and optionally the similarities and/or differences therebetween; and/or other types of component characteristics. In an embodiment, categories of components may be delineated based on the chemical structures of components, the feedstock sources of components, and the production processes of components. The component categories may be delineated, identified, or otherwise determined by using one or more manual techniques, computer-aided techniques, machine-learning, and/or artificial intelligence techniques, for example, and the component categories (e.g., at the same level of categorical nesting) may be mutually exclusive categories, in some implementations. In some embodiments, the component categories may be determined based on various different sources, tests, and/or assessments, such as the International Standards Organization (ISO) 16128 Nationally Derived (ND) ingredient data or data generated by one or more similar suitable globally recognized, standard methods of measuring ingredient naturality at the atomic level; data indicative of the biodegradation or the degradation of the materials though biotical (e.g., biotic) processes (e.g., as measured by the Organisation for Economic Co-operation and Development (OECD) Guidelines for the Testing of Chemicals, Section 3-Environmental fate and behavior,” through simulations, and/or by utilizing other or similar suitable sets of modeling and/or internationally agreed-upon testing methods), contents of the Carbon Disclosure Project (CDP) Supply Chain Green House Gas emissions reports, data provided by the ecoinvent Association and/or from other life cycle inventory databases, etc. In an embodiment, the component category e-impact ratings generator engine 102 of the system 100 may execute to identify, discover, delineate, distinguish between, or determine at least a portion of the component categories and/or to assign various components to respective component categories.

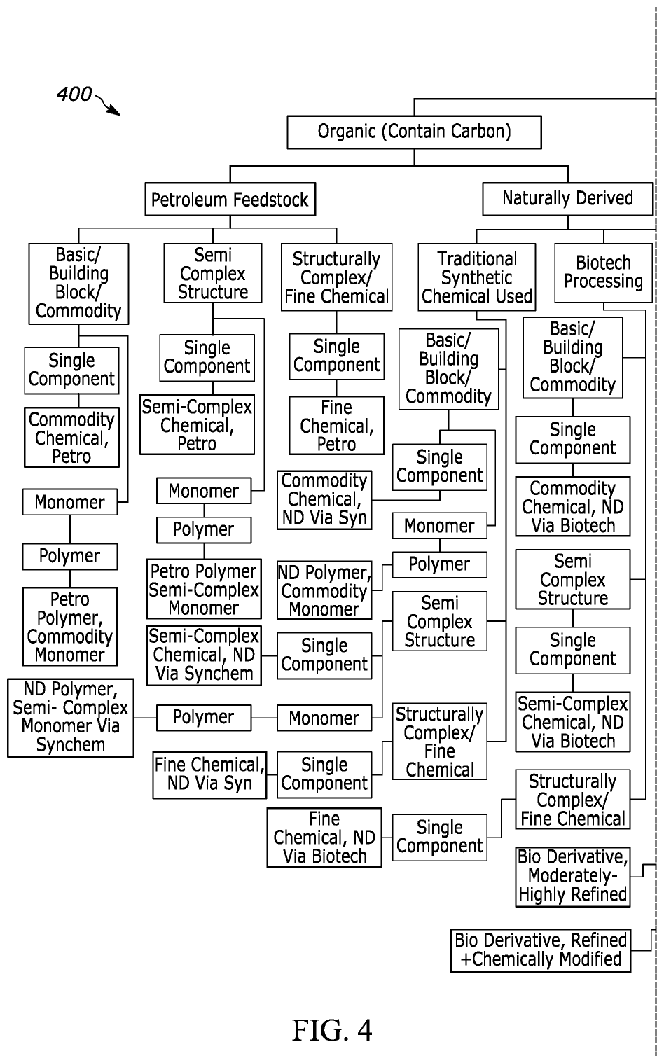

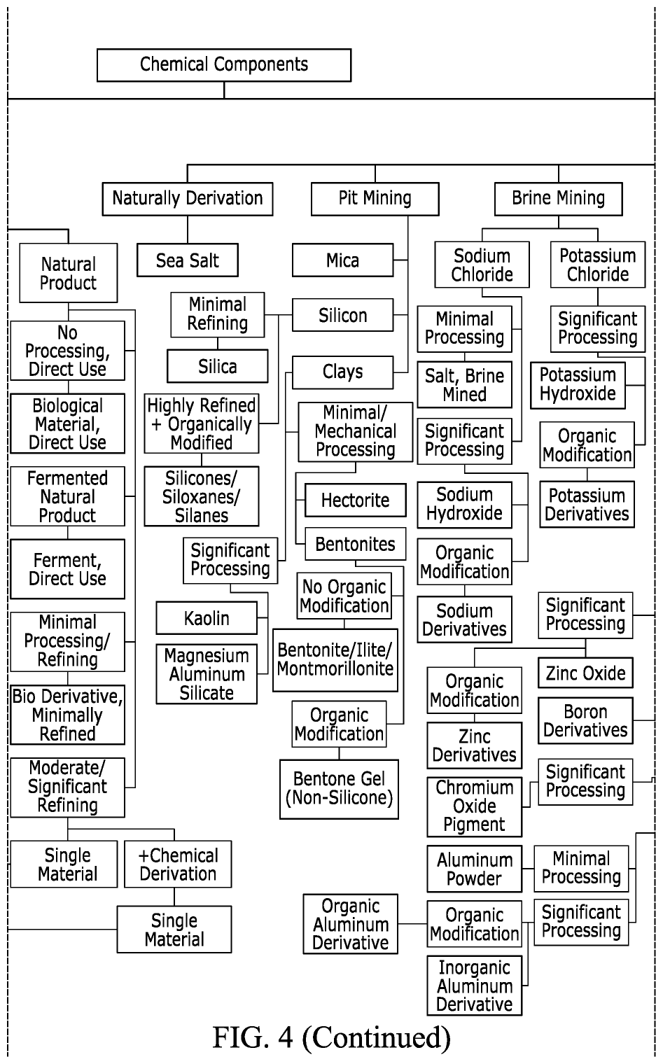

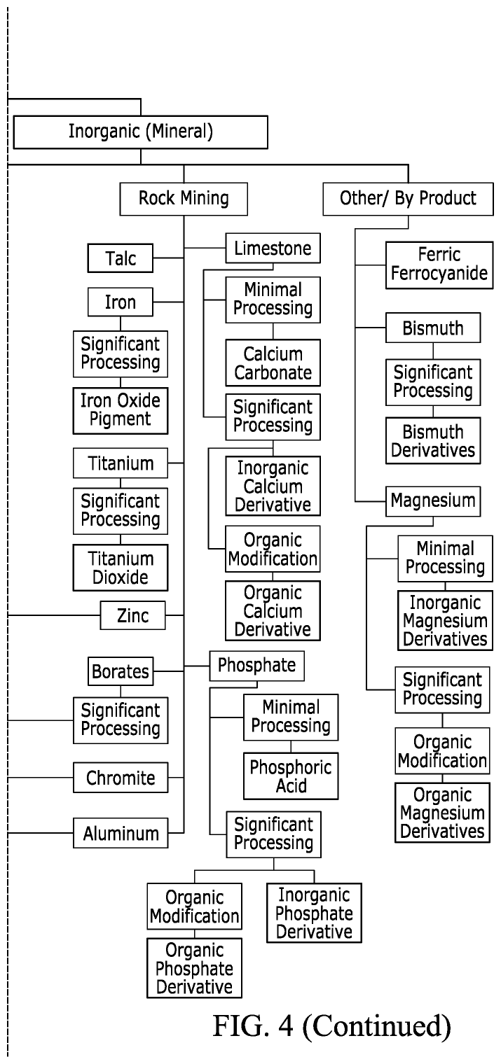

FIG. 4 depicts an example of a categorization structure or framework 400 of a plurality of component categories, including several levels of nested sub-categories. The categorization structure 400 may be at least partially generated and/or utilized by the system 100 and/or by embodiments of any of the methods and techniques described herein. It is noted that the categorization structure 400 shown in FIG. 4 is only one of many possible categorization structures, frameworks, or organizations of component categories and sub-categories and is not meant to be limiting. Further, for ease of reading herein and not for limitation purposes, the term “component category” is utilized herein to generally and categorically refer to a “component category and/or sub-category, where a sub-category may be a nested sub-category” or similar. As shown in FIG. 4, the example structure 400 of chemical component categories and corresponding levels of sub-categories is described in outline form below and includes:
A. Organic (e.g., contains carbon)
   1. Petroleum Feedstock
      a. Basic/Building Block/Commodity
         1. Single Component
         a. Commodity Chemical, Petroleum
         2. Monomer
         a. Polymer
         i. Petroleum Polymer, Commodity Monomer
      b. Semi Complex Structure
         1. Single Component
         a. Semi-complex Chemical, Petroleum
         2. Monomer
         a. Polymer
         i. Petroleum Polymer, Semi-Complex Monomer
      c. Structurally Complex/Fine Chemical
         1. Single Component
         a. Fine Chemical, Petroleum
   2. Naturally Derived (ND)
      a. Synthetic Chemical Chemistry Used
         1. Basic/Building Block/Commodity
         a. Single Component
         i. Commodity Chemical, ND via synthetic chemical processing (e.g., and not via biotechnology or fermentation)
         b. Monomer
         i. Polymer
         1. ND Polymer, Commodity Monomer via synthetic chemical processing (e.g., and not via biotechnology or fermentation)
         2. Semi Complex Structure
         a. Single Component
         i. Semi-complex Chemical, ND via synthetic chemical processing (e.g., and not via biotechnology or fermentation)
         b. Monomer
         i. Polymer
         1. ND Polymer, Semi-Complex Monomer via synthetic chemical processing (e.g., and not via biotechnology or fermentation)
         3. Structurally Complex/Fine Chemical
         a. Single Component
         i. Fine Chemical, ND via synthetic chemical processing (e.g., and not via biotechnology or fermentation)
      b. Biotechnological Processing
         1. Basic/Building Block/Commodity
         a. Single Component
         i. Commodity Chemical, ND via biotechnology or fermentation (e.g., and not via synthetic chemical processing)
         2. Semi Complex Structure
         a. Single Component
         i. Semi-complex Chemical, ND via biotechnology or fermentation (e.g., and not via synthetic chemical processing)
         3. Structurally Complex/Fine Chemical
         a. Single Component
         i. Fine Chemical, ND via biotechnology or fermentation (e.g., and not via synthetic chemical processing)
      c. Natural Product
         1. No Processing, Direct Use
         a. Biological Material, Direct Use
         2. Fermented Natural Product
         a. Ferment, Direct Use
         3. Minimal Processing/Refining
         a. Bio Derivative, Minimally Refined
         4. Moderate/Significant Refining
         a. Single Material
         i. Bio Derivative, Moderately to Highly Refined
         b. Chemical Derivation
         i. Single Material
         1. Bio Derivative, Refined and Chemically Modified
B. Inorganic (e.g., minerals)
   1. Natural Derivation
      a. Sea Salt
   2. Pit Mining
      a. Mica
      b. Silicon
         1. Minimal Refining
         a. Silica
         2. Highly Refined and Organically Modified
         a. Silicones/Siloxanes/Silanes
      c. Clays
         1. Minimal/Mechanical Processing
         a. Hectorite
         b. Bentonites
         i. No Organic Modification
         1. Bentonite/Ilite/Montmorillonite
         ii. Organic Modification
         1. Bentone Gel (non-silicone)
         2. Significant Processing
         a. Kaolin
         b. Magnesium Aluminum Silicate
   3. Brine Mining
      a. Sodium Chloride
         1. Minimal Processing
         a. Salt, Brine Mined
         2. Significant Processing
         a. Sodium Hydroxide
         b. Organic Modification
         i. Sodium Derivatives
      b. Potassium Chloride
         1. Significant Processing
         a. Potassium Hydroxide
         b. Organic Modification
         i. Potassium Derivatives
   4. Rock Mining
      a. Talc
      b. Iron
         1. Significant Processing
         a. Iron Oxide Pigment
      c. Titanium
         1. Significant Processing
         a. Titanium Dioxide
      d. Zinc
         1. Significant Processing
         a. Organic Modification
         i. Zinc Derivatives
         b. Zinc Oxide
      e. Borates
         1. Significant Processing
         a. Boron Derivatives
      f. Chromite
         1. Significant Processing
         a. Chromium Oxide Pigment
      g. Aluminum
         1. Minimal Processing
         a. Aluminum Powder
         2. Significant Processing
         a. Organic Modification
         i. Organic Aluminum Derivative
         b. Inorganic Aluminum Derivative
      h. Limestone
         1. Minimal Processing
         a. Calcium Carbonate
         2. Significant Processing
         a. Organic Modification
         i. Organic Calcium Derivatives
         b. Inorganic Calcium Derivatives
      i. Phosphate
         1. Minimal Processing
         a. Phosphoric Acid
         2. Significant Processing
         a. Organic Modification
         i. Organic Phosphate Derivative
         b. Inorganic Phosphate Derivative
   5. Other/Byproduct
      a. Ferric Ferrocyanide
      b. Bismuth
         1. Significant Processing
         a. Bismuth Derivatives
      c. Magnesium
         1. Minimal Processing
         a. Inorganic Magnesium Derivatives
         2. Significant Processing
         a. Organic Modification
         i. Organic Magnesium Derivatives

Accordingly, as each component category may include a respective set of one or more components, where each component of the category has similar environmentally-impacting characteristics of its cradle-to-gate production processing, an e-impact rating of a component category may be indicative of an environmental impact generated by utilizing any component of the group of components of the component category in producing an ingredient of personal care products. In an example, the e-impact rating of a component category may be indicative of an environmental impact that is posed by, inherent to, resulting from, brought forth by, produced by, and/or otherwise generated by waste that is generated (e.g., cradle-to-gate) from utilizing any one or more components of the component category in producing one or more ingredients of personal care products. Generally speaking, waste that is generated in producing ingredients of personal care products may include solvents, biomass, removed impurities, catalysts, reaction byproducts, unreacted starting materials, auxiliary agents, reaction aids, greenhouse gas emissions, waste that is landfilled, and/or other types of waste that are not otherwise upcycled, composted, or otherwise transformed into (relatively more) environmentally benign byproducts.

The e-impact ratings of various component categories may be discovered, determined, or otherwise generated by the component category e-impact ratings generator engine 102 of the system 100, for example. In an embodiment, the e-impact rating of a component category may be an average of the individual component e-impact ratings of two or more components included in the component category, e.g., as stored in the component e-impact ratings data store 120 and/or as determined in real-time by the component e-impact ratings generator 108. In an embodiment, the e-impact rating of a component category may be an average of the individual component e-impact ratings of an entirety of the components that are included in the component category (e.g., as known to at least a portion of the system 100, which may include an entirety of the components indicated in the component e-impact ratings data store 120 and optionally in the other data stores 118 and 122 of the system 100). The averages of the individual component e-impact ratings utilized to determine component category e-impact ratings may be weighted averages, if desired, and the weighting of the individual component e-impact ratings to determine the averages may utilize any desired weighting technique. For example, a weighting technique may be indicative of respective levels, amounts, or strengths of alignment or association of individual component e-impact ratings with one or more of the component characteristics based on which the component categories are defined or delineated (e.g., chemical structures, feedstock procurement procedures, production processing, and/or other component characteristics that affect or impact environmental sustainability, as discussed elsewhere within this disclosure). To illustrate, an example factor which may affect the relative strengths of alignment/association of the individual component e-impact ratings with the one or more component characteristics corresponding to component category delineation may be whether production processes are optimized for target yields and/or rates of production of ingredients in which corresponding components are utilized, and/or the extent to which the production processes are optimized. Another example factor which may affect the relative strengths of alignment/association of the individual component e-impact ratings may be how many and/or which particular steps, phases, or stages of cradle-to-gate production processing are considered or utilized in generating the individual component e-impact ratings. Of course, other factors may be possible and utilized.

Accordingly, a component which is newly introduced to the system 100 (e.g., that is not previously reflected in the any of the data stores 118, 120, 122 of the system 100) may be assigned (e.g., by the component category e-impact ratings generator 102) to a particular component category of the plurality of component categories based on the respective level or strengths of association of similarity of the delineation/grouping characteristics of the newly-introduced component with respect to the respective delineation/grouping characteristics of each of the component categories, and the newly-introduced component may have, as its initial e-impact rating, the e-impact rating of the particular component category to which the newly-introduced component was assigned. Of course, the initial e-impact rating of the newly-introduced component may be determined by other techniques, such as by using a present-day Green Score tool or other technique.

With further regard to the component e-impact ratings generator engine 108, in embodiments, the component e-impact ratings generator engine 108 may adjust or alter the initial component e-impact ratings of the plurality of candidate components based on the range of the initial component e-impact ratings, or in another relative manner if desired. Accordingly, the adjusted or altered initial component e-impact rating of a first candidate component of the plurality of candidate components may be relative or with respect to the adjusted or altered initial component e-impact rating of one or more other candidate components of the plurality of candidate components.

In an embodiment, the adjustment or alteration of the initial component e-impact ratings of the plurality of candidate components may include weighting the initial component e-impact ratings in a relative manner among the plurality of candidate components. The weightings utilized for the purposes of relativity among the plurality of candidate components may be a different set of weightings than those utilized for determining component category e-impact ratings, and may be determined independently. At any rate, in this embodiment, the component e-impact ratings generator engine 108 may determine and assign respective weights or scores to the initial component e-impact ratings for relativity purposes using one or more desired weighting techniques. One example of such a weighting technique may be a weighting technique that utilizes an arbitrary linear, inverse, or exponential equation, e.g., to normalize the plurality of initial component e-impact ratings. In another example, a weighting technique may set an arbitrary e-impact rating “cutoff” so that all initial component e-impact rating values indicating a less preferred or less desirable environmental impact resulting from waste generation (e.g., above or below the cutoff, depending on the implemented directionality of the rating values) receive a particular score or weight, or so that all initial component e-impact rating values indicating a more preferred or more desirable environmental impact resulting from waste generation relative to the cutoff receive a certain score or weight. In yet another example, a weighting technique may set arbitrary e-impact rating ranges that correspond to respective numerical scores to thereby yield a non-continuous set of scores or weights for the plurality of initial component e-impact ratings. In still another example, a weighting technique may set respective scores or weights for the plurality of initial component e-impact ratings based on a distribution analysis of the range of the plurality of initial component e-impact ratings. Of course, other weighting techniques may be additionally or alternatively utilized to determine and assign respective weights or scores to the plurality of initial component e-impact ratings for relativity purposes.

In an additional or alternate embodiment, the adjustment or alteration of the initial component e-impact ratings of the plurality of candidate components may include a scaling of the initial component e-impact ratings. For example, the component e-impact ratings generator engine 108 may scale the initial component e-impact ratings of the plurality of candidate components. Such a scaling may include a transformation, such as a logarithmic transformation or another type of normalizing transformation, or may utilize any other type of suitable scaling technique. In some cases, the scaling of the initial component e-impact ratings may include or utilize a scoring cutoff, e.g., in a manner as described above for weighting-related cutoffs. At any rate, the scaling may be based on the range of the initial component e-impact ratings of the plurality of candidate components, and/or the scaling may be implemented based on one or more requirements of the desired representation of the initial e-impact ratings (for example, a scale of zero to five, zero to 10, zero to 100, zero to 1000, etc.) and the desired directionality of the scale. For instance, in some implementations, a scaled initial component e-impact rating of zero may indicate zero or negligible environmental impact due to utilizing a corresponding component in ingredients of personal care products, and thus may indicate that the corresponding component is most environmentally preferred among a set of components. In other implementations, a scaled initial component e-impact rating of zero may indicate a maximum negative environmental impact (among a set of components) due to utilizing a corresponding component in ingredients of personal care products, and thus may indicate that the corresponding component is least environmentally-preferred among the set of components. In some implementations, a distributional analysis of the range of initial component e-impact ratings may be performed, and a normalization transformation or equation may be selected based on the results of the distributional analysis, e.g., to enable increased scoring differentiation between initial component e-impact rating values that correspond to lesser waste generation impacts. For some distributions, such as for distributions in which the range of distribution is large and/or skewed, the selected normalization equation may be a logarithmic normalization equation; however, other types of normalization equations may be selected and utilized as desired. Generally speaking, with respect to the scaling of the initial component e-impact ratings, a transformation (logarithmic or otherwise) may be selected in accordance with the desired requirement(s) of the desired scaling representation of the initial component e-impact ratings (e.g., a scale of zero to five, zero to 10, zero to 100, zero to 1000, etc.) and the desired directionality of the scale.

Turning now to the e-hazard adjustment generator engine 105, the e-hazard adjustment generator 105 may execute to discover, determine, or otherwise generate respective e-hazard adjustments of each component category included in the plurality of component categories and the component category e-hazard adjustments may be stored in the component category e-hazard adjustments data store 118. Typically, but not necessarily, the e-hazard adjustment generator 105 may generate the respective e-hazard adjustment of a component category independently of the determination of the e-impact rating of the component category and/or of the determination of the respective e-impact ratings of any individual components included in the component category. Accordingly, in the system 100, the e-hazard adjustment of a component category may be generated by the e-hazard adjustment generator 105 prior to, after, or in parallel to the determination, by the component e-impact ratings generator 108 and or by the component category e-impact ratings generator 102, of the initial and/or modified e-impact rating of any of the individual components included in the component category and of the component category itself. Said another way, typically the e-hazard adjustment generator 105 may not utilize the e-impact rating of the component category or the respective e-impact ratings of the components of the component category to generate the e-hazard adjustment of the component category, so that the e-hazard adjustment of the component category may be generated independently of the e-impact ratings of the component category and the initial e-impact ratings of any individual components included in the component category.

Generally, an e-hazard adjustment of a component category may be indicative of the amount or level of environmental hazard posed by (e.g., inherent to, resulting from, brought forth by, produced by, and/or generated by) the waste produced by utilizing, e.g., from cradle-to-gate, components of the component category to produce personal care products. That is, the e-hazard adjustment of a component category may be indicative of the amount or level of environmental hazard posed by the waste produced by utilizing of any of the components included in the component category, generally, rather than by utilizing any specific one component included in the component category.

As previously discussed, the different component categories may be delineated based on different environment-affecting characteristics and/or combinations thereof, for example, chemical structures of components, feedstock sources of components, and/or production processes of the components (and in some cases, other additional characteristics). Accordingly, in embodiments, the e-hazard adjustments of different component categories may be scaled across the range of environment-affecting characteristics or the range of combinations of environment-affecting characteristics so that the e-hazard adjustments may be indicative of the relative amounts of environmental hazard posed by generated waste across the different component categories. For example, consider a first component category corresponding to semi-complex molecules derived from petroleum feedstock and a second component category corresponding to semi-complex molecules which, when processed into personal care product ingredients, generate waste that is at least partially renewable or recyclable via fermentation into environmentally benign by-products. The respective e-hazard adjustments of the first component category and the second component category may indicate that components of the second category are more environmentally-preferred than components of the first category to process into ingredients of personal care products, as the amount of cradle-to-gate hazardous waste generated by utilizing components of the second category in ingredients of personal care products as indicated by the e-hazard adjustment of the second category is less than the amount of cradle-to-gate hazardous waste generated by utilizing components of the first category as indicated by the e-hazard adjustment of the first category. Said another way, the e-hazard adjustments of different component categories may be relative to one another, e.g., across the range or entirety of a set of component categories. The e-hazard adjustment generator engine 105 may determine the respective e-hazard adjustments of the different categories of components by using one or more computer-aided techniques, machine-learning, and/or artificial intelligence techniques, for example, and/or optionally by using one or more manual techniques.

Because the e-hazard adjustment of a component category may be indicative of an amount or level of environmental hazard posed by waste that is generated across cradle-to-gate production processing of the category's components, the e-hazard adjustment of a component category may be indicative of the amount or level of waste that is generated from multiple phases or stages of production processing, e.g., from the initial procurement of feedstock corresponding to the category's components to the production of the end-product ingredients of personal care products in which the category's components are included. The e-hazard adjustment of the component category may correspond to the cradle-to-gate production processing of components of the component category into a single, specific ingredient, or may correspond to the cradle-to-gate production processing of the components of the component category into any ingredient of a group of ingredients. Unlike present-day E-factors which typically are indicative of the waste generated by only the chemical processing of a specific, individual component, the component category e-hazard adjustment which is generated by the e-hazard adjustment generator 105 may be indicative of the amount or level of environmental hazard posed by waste that is generated from multiple (e.g., two or more) stages or phases of the cradle-to-gate production processing of any component of the multiple components included in the component category into end-product ingredients of personal care products. Such production processing stages or phases may include, for example, procuring the feedstock(s), material processing (if necessary, and/or other types of initial processing) of the procured feedstock(s) and/or other starting materials, one or more stages or phases of chemical processing, purifying the output generated by the chemical processing, and/or other steps, stages, or phases that are utilized in the overall production processing of the category's components from feedstock into forms of end-product ingredients that are usable to combine with other ingredients to produce personal care end-products. In a preferred but non-limiting embodiment, the e-hazard adjustment of the component category may be indicative of the amount or level of environmental hazard posed by waste that is generated from at least the production processing steps, stages, or phases of the procuring of the feedstock(s), the processing of the procured feedstock(s) into components of the category, and the disposal and/or mitigation of the waste generated by the procuring of the feedstock and the processing of the procured feedstock into the components of the category.

Accordingly, at least a portion of the e-hazard adjustment of a component category may be indicative of an amount or level of environmental hazard posed by waste that is generated by procuring feedstock of components of the category from one or more corresponding feedstock sources. Such feedstock procurement techniques may include, for example, mining, harvesting, refining, agricultural production, mineral processing, chemical synthesis, chemical modification, biological derivation, fermentation, enzymatic catalysis, another type of procurement technique that utilizes a biological organism or convergent synthesis, and/or other procurement techniques. Additionally or alternatively, at least a portion of the e-hazard adjustment of a component category may be indicative of an amount or level of environmental hazard posed by waste that is generated by processing the procured feedstock into components of the category and/or by processing the components of the category into ingredients of personal care products, where such processing activities may include, for example, the processing of starting and/or intermediate materials (e.g., material processing and/or other types of initial processing), equipment cleaning, one or more phases of chemical processing, purifying, etc. Further, at least a portion of the e-hazard adjustment of a component category may be indicative of an amount or level of environmental hazard posed by waste resulting from disposing of and/or mitigating the waste that is generated by at least the procuring of the feedstocks of the components of the category and the processing of the feedstock into the components of the category, and optionally resulting from other steps/phases of cradle-to-gate production processing as well. Such waste disposal and/or waste mitigation techniques may include, for example, gasification, composting, utilization of waste as microbial feedstock, recycling and/or upcycling waste into materials that are less or not environmentally hazardous, processing through aqueous waste treatment plants, landfilling, and/or other waste disposal and/or mitigation techniques. Generally speaking, the e-hazard adjustment of a component category may be based on two or more factors corresponding to the production processes of ingredients, where such factors may include reagents, auxiliaries, catalysts, reaction mechanisms, reaction aids, solvents, reaction byproducts, removed impurities, unreacted starting materials, carbon or greenhouse gas emissions, solvent recovery, solvent loss, purification, equipment cleaning processes, manufacturing of starting materials, neutralization, byproduct recovery, and/or hazard quality of the waste due to the utilization of components of the category in ingredients of personal care products related to the cradle-to-gate production processing related to the components. As such, in an example implementation, the e-hazard adjustment may be indicative of a net amount or level of environmental hazard posed by waste resulting from the two or more factors corresponding to the production processes of ingredients, where each individual factor may have a respective negative or a positive effect on the net amount of the environmental hazard. In an embodiment, the e-hazard adjustment of the component category may be based (all of) the reagents, the auxiliaries, the catalysts, the reaction mechanisms, the reaction aids, the solvents, the reaction byproducts, the removed impurities, the unreacted starting materials, the carbon or greenhouse gas emissions, the solvent recovery, the solvent loss, the purification, the equipment cleaning processes, the manufacturing of starting materials, the neutralization, the byproduct recovery, and the hazard quality of the waste due to the utilization of the component in the ingredients of personal care product, e.g., as related to the cradle-to-gate production processing of feedstocks into end-product ingredients of personal care products.

Further, the e-hazard adjustment of a component category may be expressed as a penalty or a bonus, e.g., with respect to the impact on environmental sustainability. For example, component categories that generally produce less environmentally-hazardous waste across cradle-to-gate production may have e-hazard adjustments that are bonuses which reflect the relative increase in positive effects on environmental sustainability (e.g., as compared to using components of another component category), and component categories that generally produce more environmentally-hazardous waste across cradle-to-gate production may have e-hazard adjustments that are penalties to reflect the increased adverse effects on environmental sustainability (e.g., as compared to using components of another component category). The e-hazard adjustment may be expressed in any suitable format, for example, as an additive or a subtractive adjustment, as a factor or multiplier, as a function, etc.

Returning again to the component e-impact ratings generator 108, in an embodiment, the component e-impact ratings generator 108 may modify the initial e-impact rating of a candidate component (which may have been adjusted, altered, or scaled, e.g., relative to the range of initial e-impact ratings of the plurality of candidate components, such as described elsewhere within this document) based on the e-hazard adjustment of the candidate component. The component e-impact ratings generator 108 may modify an obtained initial e-impact rating of a candidate component, or may modify an adjusted, altered, or scaled (e.g., relative to the range of initial e-impact ratings of the plurality of candidate components) initial e-impact rating of the candidate component. The modification may be based on the format in which the e-hazard adjustment is represented. For example, when the e-hazard adjustment is an additive adjustment, the component e-impact ratings generator 108 may add the e-hazard adjustment of the candidate component to the initial e-impact rating of the candidate component; when the e-hazard adjustment is a subtractive adjustment, the component e-impact ratings generator 108 may subtract the e-hazard adjustment of the candidate component from the initial e-impact rating of the candidate component; when the e-hazard adjustment is a factor or multiplier, the component e-impact ratings generator 108 may multiply the e-hazard adjustment and the initial e-impact rating of the candidate component; when the e-hazard adjustment is a function, the component e-impact ratings generator 108 may apply the function to the initial e-impact rating of the candidate component; etc. Generally speaking, the application of the e-hazard adjustment to the initial e-impact rating of the candidate component may generate an enhanced e-impact rating of the candidate component, which the component e-impact ratings generator 108 may store into the component enhanced e-impact ratings data store 122.

The ingredient selector engine 110 may compare two or more respective component enhanced e-impact ratings (e.g., as stored in the component enhanced e-impact ratings data store 122) of two or more candidate components. In an embodiment, based on the comparison, the ingredient selector engine 110 may select at least one of the two or more candidate components for utilizing in an ingredient of a personal care product. Generally speaking, the ingredient selector engine 100 may select candidate components that are more environmentally preferred (e.g., have more favorable enhanced e-impact ratings) as compared to other candidate components. In an embodiment, the ingredient selector engine 110 may additionally or alternatively select, based on the comparison of the component enhanced e-impact ratings, at least one ingredient for utilizing in a formulation of the personal care product, where the ingredient includes or is otherwise produced using one or more candidate components that are more environmentally-preferred (e.g., have more favorable enhanced e-impact ratings) as compared to other candidate components. The system 100 may utilize the selected component and/or the selected ingredient to produce a formulation for the personal care product, for example. Additionally or alternatively, the system 100 may utilize the selected component and/or the selected ingredient to produce or manufacture the personal care product. In an embodiment, the ingredient selector engine 110 may send an indication of the selected component(s) and/or of the selected ingredient(s) to a user interface, to another computing device or system, and/or to another computer application executing on another computing device or system. For example, the ingredient selector engine 110 may send (e.g., via the one or more communication interfaces 130) the indication of the selected component(s) and/or of the selected ingredient(s) to a formulation system, an automation system, a manufacturing system, a control system, an inventory system, a supply chain management system, and/or other types of systems associated with producing personal care products to thereby instruct the recipient system(s) to utilize the selected component(s) and/or selected ingredient(s) in producing formulations of personal care products and/or in producing or manufacturing personal care end-products. Additionally or alternatively, the ingredient selector engine 110 may send the indication of the selected component(s) and/or of the selected ingredient(s) to a local user interface 132 and/or to a remote user interface (e.g., via the communication interfaces 130). In implementations in which the system 100 is included in a formulation, automation, manufacturing, control, inventory, or supply management system (or vice versa), the ingredient selector engine 100 may send an indication of the selected component(s) and/or of the selected ingredient(s) to another application executing within the integral system, e.g., to thereby cause the selected ingredient(s) to be utilized in the production or manufacturing of a personal care product.

Additionally, in some scenarios, the enhanced e-impact ratings of components may be utilized to determine e-impact ratings of formulations of ingredients of personal care products and/or of the ingredients themselves. For example, an e-impact rating of formulation of an ingredient of personal care products may be determined at least partially based on the respective enhanced e-impact ratings of components selected for and/or otherwise included in the formulation. Similarly, an e-impact rating of an ingredient of personal care products may be determined at least partially based on the respective enhanced e-impact ratings of components of a formulation utilized to produce the ingredient.

The enhanced e-impact ratings of components have been demonstrated to be more accurate and thus more useful than present-day Green Scores and/or E-factors. For example, the enhanced e-impact rating incorporates or otherwise takes into account the environmental impact of hazardous waste generated from utilizing a component in ingredients of personal care products (for example, by quantifying environmental hazard due to generated waste), while present-day Green Scores generally do not consider the environmental impact of hazardous waste at all. Indeed, in view of the present disclosure, the enhanced e-impact rating addresses three additional green chemistry principles that are typically not considered by at least some present-day Green Scores, specifically, the green chemistry principles of waste reduction, elimination, or prevention; maximization of atom economy; and less hazardous chemical synthesis.

Further, the enhanced e-impact rating also takes into account the environmental impact of hazardous waste generated from multiple, if not all, cradle-to-gate production processes related to utilizing a component in personal care products (e.g., including feedstock procurement processes, material processing of feedstock and other initial materials, purification, etc.) in a standardized manner. Present-day E-factors typically only address certain chemical processes within the full cradle-to-gate processing of feedstocks into ingredients, and present-day E-factors are not standardized. Thus, present-day E-factors are not and cannot be incorporated into present-day Green Score tools in any meaningful manner. However, as the enhanced e-impact ratings of the present disclosure are based on categorizations of components (e.g., based on at least chemical structures, feedstock sources, and production processes) rather than based on individual components, and as in some implementations the enhanced e-impact ratings may be scaled relative to one another, the resulting enhanced e-impact ratings have built-in standardization and thus are more robust, comprehensive, and useful as compared to present-day Green Score tools and E-factors. Still, the standardization of the enhanced e-impact ratings may easily indicate the relativity of respective environmental impacts among a plurality of candidate components, thus providing the ability to more completely and quickly assess the relative environmental impacts of utilizing different candidate components in personal care product ingredients, as well as providing the ability to accurately and quickly assess the environmental impacts of using newly-introduced components with a greater levels of confidence and accuracy while expending less resources (e.g., less computing processing and/or memory resources) than presently-used techniques. Generally speaking, the techniques of the present disclosure allow enterprises to select and utilize more environmentally-preferred components to process into or otherwise utilize in end-product ingredients of personal care products in a resource-efficient manner to thereby reduce, minimize, and in some cases eliminate a majority of undesirable environmental impacts resulting therefrom.

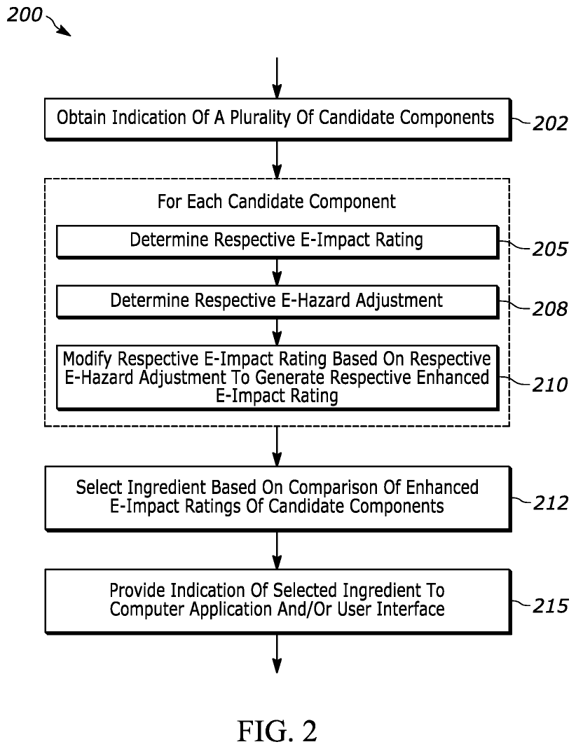

FIG. 2 depicts a flow diagram of an example method 200 for producing personal care products with environmentally-preferred ingredients. The method 200 may operate as a stand-alone method, in conjunction with other methods described herein, such as the method 300, and/or in conjunction with embodiments of at least a portion of the system 100 of FIG. 1 and optionally of any one or more devices or systems related thereto, and/or with other systems, processors, databases and/or devices. For example, the component category e-impact ratings generator engine 102, the e-hazard adjustment generator engine 105, the component e-impact ratings generator engine 108, and/or the ingredient selector engine 110 may execute at least a portion of the method 200. In some embodiments, at least a portion of the method 200 may execute in conjunction with one or more other portions of the system 100, such as with one or more of the data stores 118-125 and/or the instructions 128. For ease of discussion and not for limitation purposes, the method 200 is discussed with simultaneous reference to FIG. 1. Further, the method 200 may include additional or alternate steps other than those described with respect to FIG. 2, in embodiments.

At a block 202, the method 200 may include obtaining an indication of a plurality of candidate components (e.g., a plurality of candidate chemical components) for an ingredient of a personal care product. In an embodiment, the indication of the plurality of candidate components may be at least partially obtained via the user interface(s) 132 of the system 100, the communication interface(s) 130 of the system 100, and/or by accessing a database of components, such as the data store 125. In an example, the indication of the plurality of candidate components may indicate a subset of an entirety of the components indicated in the data store 125, where the subset may be determined based on one or more criteria provided via the user interface 132, the communication interface 130, and/or one or more executing engines and/or computer-executable instructions 102, 104, 108, 110, or 128 of the system 100.

At a block 205, the method 200 may include determining a respective initial e-impact rating of each candidate component included in the plurality of candidate components, where the respective e-impact rating of each candidate component may be indicative of an environmental impact of utilizing the candidate component in ingredients of personal care products. In some implementations, the respective e-impact rating of each candidate component may be at least partially indicative of an environmental impact of waste generated from utilizing the candidate component in ingredients of personal care products. In an embodiment, the determining 205 of the respective initial e-impact ratings of the plurality of candidate components may include obtaining at least one of the respective initial e-impact ratings from the component e-impact ratings data store 120. In an embodiment, the determining 205 of the respective initial e-impact ratings of the plurality of candidate components may include applying a present-day Green Score tool to characteristics or other information about the one or more of the candidate components and obtaining the respective initial e-impact ratings of the one or more of the candidate components via the Green Score tool output.

In an embodiment, the determining 205 of the respective initial e-impact rating of a particular candidate component (of the plurality of candidate components) may include determining an e-impact rating of a component category in which the particular candidate component is included and assigning the e-impact rating of the component category as the initial e-impact rating of the particular candidate component. The e-impact rating of a component category may be indicative of an environmental impact of utilizing any one or more of the components included in the component category in the ingredients of personal care products. In some implementations, the respective e-impact rating of the component category may be at least partially indicative of an environmental impact of waste generated from utilizing any one or more of the components of the component category in ingredients of personal care products.

Accordingly, in some implementations, the method 200 may include categorizing each candidate component into a respective component category included in the plurality of component categories. The categorization of each candidate component may be based on one or more characteristics of the candidate component, such as a chemical structure of the candidate component, one or more feedstock sources of the candidate component, and/or one or more production processes of the each candidate component, in an embodiment. In some embodiments, the method 200 may include determining, defining, or otherwise identifying at least some of the component categories themselves, such as in manners described elsewhere within this document. In an embodiment, an overall categorization structure or framework, an example of which is depicted in FIG. 4, may be determined or defined based on similarities and/or differences between various chemical structures of components, similarities and/or differences between various feedstock sources and/or types of feedstock sources of components, and/or similarities and/or differences between various processes or types of processing (e.g., materials, mechanical, chemical, etc.) used to produce ingredients from feedstock sources. For instance, the determining, defining, or otherwise identifying at least some of the component categorizations may include classifying the chemical structures of components, classifying feedstock sources of components, and/or classifying the production processes utilized to process components into ingredients of personal care products.

At any rate, the e-impact rating of the component category in which the particular candidate component is included may be based on an average of e-impact ratings of a group of components included in the component category in which the particular candidate component is included. As such, in some embodiments, the method 200 may include determining the average of the e-impact ratings of the group of components included in component category of the particular candidate component. Typically, but not necessarily, the group of components based on which the average is determined may include a majority of the components included in the component category, and in some cases the group of components may include all of the components included in the component category. In some implementations, the average of the e-impact ratings of the group of components may be a weighted average. Accordingly, in some embodiments, the method 200 may include determining the weighting technique that is utilized to determine the weighted average. The weighting technique may include one or more of the weighting techniques described elsewhere in this document, for example.

Additionally or alternatively, the determining 205 of the initial e-impact rating of the particular candidate component may include determining a level or amount of environmental hazard posed by waste that is generated from utilizing the particular candidate component in ingredients of personal care products. For example, the initial e-impact rating of the particular candidate component may be determined 205 based on the level or amount of environmental hazard posed by the net waste that is generated across multiple different stages or phases (e.g., cradle-to-gate stages or phases) in the production processing of the particular candidate component into one or more ingredients of personal care products. Such production processing stages or phases based on which the initial e-impact rating of the particular candidate component may be determined 205 may include, for example, procuring feedstock(s) corresponding to the particular candidate component; processing the procured feedstock(s) into the particular candidate component; chemically processing the particular candidate component (in some cases in conjunction with other components through several rounds of chemical processing), purifying the chemically-processed particular candidate component, and/or disposing of and/or mitigating at least a portion of the waste that is respectively generated by each production processing stage or phase contributing to the net waste. Accordingly, the waste (e.g., the net waste) that is generated by the production processing of the particular candidate component into ingredients of personal care products may include waste generated from or based on one or more factors related to the various stages or phases of production processing such as, for example, reagents, auxiliaries, catalysts, reaction mechanisms, reaction aids, solvents, reaction byproducts, removed impurities, unreacted starting materials, greenhouse gas emissions, solvent recovery, solvent loss, purification, equipment cleaning processes, manufacturing of starting materials, recycling, upcycling, gasification, composting, utilization of at least a portion of the waste as microbial feedstock, processing through aqueous waste treatment plants, landfilling, byproduct recovery, and/or hazard quality of the waste due to the utilization of the particular candidate component in ingredients of personal care products, to name a few. In embodiments, the waste (e.g., the net waste) that is generated by the production processing of the particular candidate component and/or otherwise utilizing the particular candidate component in ingredients of personal care products may include waste generated from or based on at least two of said factors. In embodiments, the waste (e.g., the net waste) that is generated by the production processing of and/or otherwise utilizing the particular candidate component in ingredients of personal care products may include waste generated from or based on a majority or even all of said factors discussed above.

Still further, in some embodiments (not shown in FIG. 2), the method 200 may include adjusting or altering the determined initial e-impact rating of the particular candidate component based on other candidate components, e.g., in a relative manner with respect to other candidate components. For example, the method 200 may include weighting the initial e-impact rating of the particular candidate component and/or scaling the initial e-impact rating of the particular candidate component (e.g., in conjunction with the initial e-impact ratings of other candidate components), for example, in manners similar to those described elsewhere within this disclosure. In an implementation, the adjusted or altered initial component e-impact rating of the particular candidate component may be indicative of the relativity of the initial e-impact rating of the particular candidate component with respect to the initial e-impact ratings of other candidate components.

At a block 208, the method 200 may include determining, for each candidate component, a respective e-hazard adjustment of a respective component category in which the candidate component is included. The respective e-hazard adjustment of the respective component category of each candidate component may be indicative of a respective amount of environmental hazard posed by waste that is generated by utilizing, in ingredients of personal care products, any one or more components of the respective component category. For example, the respective e-hazard adjustment for the respective component category may be indicative of a respective amount of environmental hazard posed by waste that is generated across multiple different stages or phases of production processing of any one or more of the components included in the respective component category of the each candidate component.

As previously discussed, the respective component category of each candidate component may be included in a plurality of categories of components used or for use in the ingredients of personal care products, where the plurality of component categories may be delineated based on chemical structures of the components, feedstock sources of the components, and production processes of the components (and optionally may be delineated additionally based on other characteristics of the components), and where each component category of the plurality of component categories may include a respective plurality of components used or for use in the ingredients of personal care products. As also discussed elsewhere herein, the stages or phases of component production processing corresponding to the determination of e-hazard adjustments may include at least the procurement of feedstocks corresponding to components, the processing of the procured feedstocks into components, and the disposal and/or mitigation of waste generated by the procurement of the feedstocks and/or the processing of the procured feedstocks into components. In some implementations, the determination of e-hazard adjustments may also be based on other stages or phases of production processing (e.g., chemical processing, purification, etc.) and any disposal and/or mitigation of waste generated by the other stages or phases, and in some implementations the determination of e-hazard adjustments may be based on all stages or phases (e.g., the entirety of cradle-to-gate stages or phases) of production processing of components into ingredients. Examples of feedstock procurement techniques may include mining, harvesting, refining, agricultural production, mineral processing, chemical synthesis, chemical modification, biological derivation, fermentation, enzymatic catalysis, and/or another type of procurement that utilizes a biological organism or convergent synthesis, to name a few. Examples of waste disposal and/or waste mitigation techniques may include recycling, upcycling, gasification, composting, utilization as microbial feedstock, processing through aqueous waste treatment plants, landfilling, and/or one or more other waste mitigation techniques.

For some component categories, the respective e-hazard adjustment is a penalty which is indicative of a greater amount of environmental hazard posed by waste that is generated by utilizing components of the component category in personal care products, e.g., as compared to the amount of environmental hazard posed by waste that is generated by utilizing components of other component categories. For some component categories, the respective e-hazard adjustment is a bonus which is indicative of a lesser amount of environmental hazard posed by waste that is generated by utilizing components of the component category in personal care products, e.g., as compared to the amount of environmental hazard posed by waste that is generated by utilizing components of other component categories. Generally speaking, the respective e-hazard adjustment of a component category may be indicative of the respective amount of environmental hazard posed by the waste that is generated by utilizing components of the component category in the ingredients of personal care products relative to amounts of environmental hazards posed by waste that is generated by utilizing components of other component categories in the ingredients of personal care products. The respective e-hazard adjustments may be expressed in any suitable format, such as by an additive or subtractive adjustment, a multiplier or factor, a function, etc.

At a block 210, the method 200 may include, for each candidate component included in the plurality of candidate components, modifying the respective initial e-impact rating of each candidate component based on the respective e-hazard adjustment corresponding to the respective component category of the candidate component, thereby generating a respective enhanced e-impact rating of each candidate component. For embodiments in which the initial e-impact ratings of the candidate components were adjusted or altered (e.g., at the block 205), the block 210 may include modifying the adjusted or altered respective initial e-impact rating of each candidate component based on the respective e-hazard adjustment corresponding to the respective component category of the candidate component. At any rate, the modifying of the initial e-impact rating (or the adjusted/altered initial e-impact rating, as the case may be) of a candidate component based on the respective e-hazard adjustment may tune or further adjust the initial e-impact rating or the adjusted/altered initial e-impact rating of the candidate component based the amount of environmental hazard that is posed by waste generated from utilizing components of the component category in which the candidate component is included to generate ingredients of personal care products, e.g., through multiple phases and stages of production processes, and in some cases, through an entirety of cradle-to-gate production processes. Thus, when an e-hazard adjustment of the candidate component's category is a penalty, the enhanced e-impact rating of the candidate component may indicate that the candidate component is less environmentally-preferred than indicated by the initial e-impact rating of the candidate component, and when an e-hazard adjustment of the candidate component's category is a bonus, the enhanced e-impact rating of the candidate component may indicate that the candidate component is more environmentally-preferred than indicated by the initial e-impact rating of the candidate component. The modifying 210 may correspond to the format in which the e-hazard adjustment of the component category is expressed. For example, when the e-hazard adjustment is an additive or subtractive adjustment, modifying the initial e-impact rating (which may an adjusted or altered initial e-impact rating) of a candidate component may include adding the respective e-hazard adjustment of a corresponding component category to or subtracting the respective e-hazard adjustment of the corresponding component category from the respective initial e-impact rating of the candidate component; when the e-hazard adjustment is a factor or a multiplier, modifying the initial e-impact rating (which may an adjusted or altered initial e-impact rating) of a candidate component may include multiplying the initial e-impact rating of the candidate component and the e-hazard adjustment of a corresponding component category; when the e-hazard adjustment is a function, modifying the initial e-impact rating (which may an adjusted or altered initial e-impact rating) of a candidate component may include applying the function to the initial e-impact rating and optionally other characteristics of the candidate component, etc. The enhanced e-impact rating of each candidate component may be stored in the component enhanced e-impact ratings data store 122, for example.

At a block 212, the method 200 may include selecting, based on a comparison of the enhanced e-impact ratings of the plurality of candidate components (e.g., as stored in the component enhanced e-impact ratings data store 122 and/or as determined in-line with the execution of the method 200), an ingredient that includes a particular component of the plurality of candidate components, where the ingredient may be utilized in a formulation of a personal care product. Generally speaking, the particular component included in the ingredient may have a more environmentally preferred enhanced e-impact rating as compared to other candidate components which may be included in the ingredient. Consequently, the selecting 212 of the ingredient may include selecting an ingredient that is produced from a more environmentally preferred component rather than from other less environmentally preferred candidate components. Additionally or alternatively, the selecting 212 of the ingredient may include selecting an ingredient that is produced from a more environmentally preferred component rather than selecting other ingredients that are processed from less environmentally preferred components. Indeed, in some embodiments (not shown in FIG. 2), the method 200 may further include determining, based on the enhanced e-impact rating of the particular component and optionally based on respective enhanced e-impact ratings of other components, a respective e-impact rating of each ingredient of one or more candidate ingredients which may be included in or otherwise processed into a personal care product (e.g., either alone or in combination with other ingredients), and the selecting 212 of the ingredient may be based on a comparison of the e-impact ratings of the one or more candidate ingredients. Additionally, in some embodiments (not shown in FIG. 2), the method 200 may include utilizing the selected ingredient in which the particular component is included in a formulation of the personal care product and/or in the manufacturing or producing of the personal care product, e.g., based on the formulation.

In some embodiments (not shown in FIG. 2), the method 200 may further include determining, based on the enhanced e-impact rating of the particular component and optionally based on respective enhanced e-impact ratings of other components, a respective e-impact rating of each formulation of one or more candidate formulations which may utilized to produce or manufacture the personal care product, and/or selecting a particular formulation of the personal care product based on a comparison of the e-impact ratings of the one or more candidate formulations. As such, the selecting of the particular formulation may include selecting a formulation that utilizes a more environmentally preferred component and/or ingredient rather than utilizes other less environmentally preferred components and/or ingredients, and/or the selecting of the particular formulation may include selecting a formulation that is more environmentally preferred as compared to other candidate formulations. The method 200 may additionally include utilizing the selected formulation to manufacture or produce the personal care product.

At a block 215, the method 200 may include providing an indication of the selected ingredient to at least one of a computer application or a user interface to thereby cause the selected ingredient to be utilized in the manufacturing of the personal care product. For example, at the block 215, the indication of the selected ingredient may be provided to one or more local user interfaces 132, and/or the indication of the selected ingredient may be provided via the communication interface(s) 130 to one or more remote user interfaces, one or more other computing devices, one or more other systems utilized in the production of personal care products (such as a formulation system, an automation system, a manufacturing system, a control system, an inventory system, a supply chain management system, etc.), and/or one or more computing applications executing on the other computing devices and/or on the other systems. In some arrangements, such as when the system 100 is integral with another system utilized in the production of personal care products (such as a formulation system, an automation system, a manufacturing system, a control system, an inventory system, a supply chain management system, etc.), at the block 215 the method 200 may include providing the indication of the selected ingredient to a computing application executing on the integral system, e.g., as represented by the engines/instructions 128, and the computing application may execute to generate a formulation and/or to control an industrial plant to produce the personal care product using the formulation, for example.

In some embodiments (not shown), the method 200 may additionally include integrating the respective e-hazard adjustment of the respective component category (e.g., the respective e-hazard adjustment determined at the block 208) with a tool that is configured and executes to evaluate, assess, and/or measure the environmental impacts of processing (e.g., production processing) of different components for use in personal care products and/or the environmental impacts of using the different components themselves in personal care products, to thereby update the tool. The tool may be a present-day Green Score tool or a similar tool, and the present-day Green Score tool (or similar) may be included in the system 100 or may be located remotely with respect to the system 100. In an example implementation, a present-day Green Score tool may be updated to utilize one or more e-hazard adjustments (e.g., as determined by the method 200) so that the updated tool is configured to evaluate, based at least on the integrated e-hazard adjustment(s), environmental impacts of waste generated from the production processing of components for use in personal care products and/or environmental impacts of utilizing the components themselves in personal care products. Consequently, scores or measurements generated by the updated tool may be at least partly based on the one or more e-hazard adjustments which were integrated with or into the updated tool.

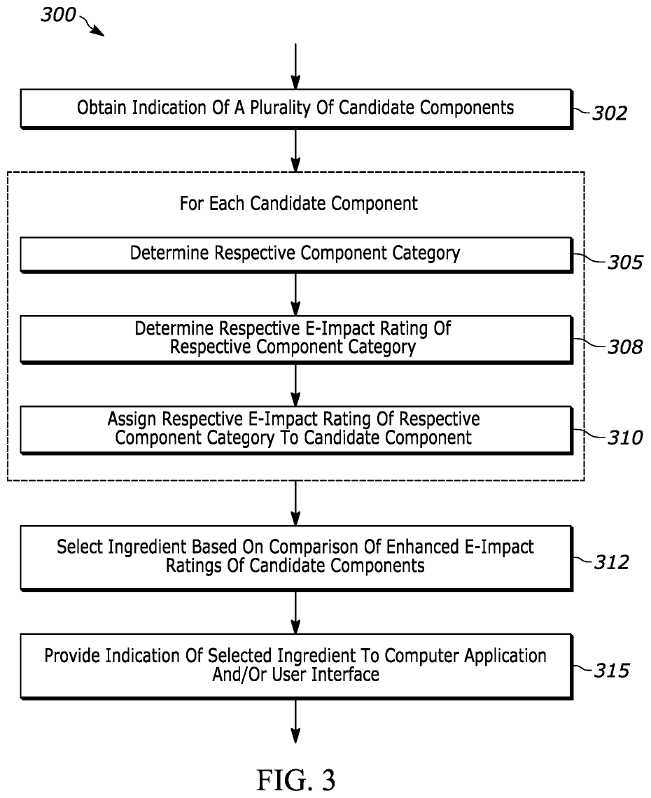

FIG. 3 depicts a flow diagram of an example method 300 for producing personal care products with environmentally preferred ingredients. The method 300 may operate as a stand-alone method, in conjunction with other methods described herein, such as the method 200, and/or in conjunction with embodiments of at least a portion of the system 100 of FIG. 1 and of any one or more components and/or devices related thereto, and/or with other systems, processors, databases and/or devices. For example, the component category e-impact ratings generator engine 102, the e-hazard adjustment generator engine 105, the component e-impact ratings generator engine 108, and/or the ingredient selector engine 110 may execute at least a portion of the method 300. In some embodiments, at least a portion of the method 300 may execute in conjunction with one or more other components of the system 100, such as with one or more of the data stores 118-125 and/or the instructions 128. For ease of discussion, and not for limitation purposes, the method 300 is discussed with simultaneous reference to FIG. 1. Further, the method 300 may include additional or alternate steps other than those described with respect to FIG. 3, in embodiments.

At a block 302, the method 300 may include obtaining an indication of a plurality of candidate components (e.g., a plurality of candidate chemical components) for an ingredient of a personal care product. In an embodiment, the indication of the plurality of candidate components may be at least partially obtained via the user interface(s) 132 of the system 100, the communication interface(s) 130 of the system 100, and/or by accessing a database of components, such as the data store 125. In an example, the indication of the plurality of candidate components may indicate a subset of an entirety of the components indicated in the data store 125, where the subset may be determined based on one or more criteria provided via the user interface 132, the communication interface 130, and/or one or more executing engines and/or computer-executable instructions 102, 104, 108, 110, or 128 of the system 100.

At a block 305, the method 300 may include determining a respective component category for each candidate component included in the plurality of candidate components. In an embodiment, at the block 305 the method 300 may include categorizing or assigning a candidate component into a respective component category of a plurality of component categories, where each component category of the plurality of component categories may include a respective one or more components used or for use in the ingredients of personal care products. The delineation of the plurality of component categories (and, in some cases, the categorizing or assigning of components to categories) may be based on chemical structures of the components (e.g., based on strengths of similarities and/or differences between the various chemical structures of components), feedstock sources of the components (e.g., based on strengths of similarities and/or differences between the various feedstock sources), and production processes or types of production processing used to produce ingredients from feedstock sources (e.g., materials processing, mechanical processing, chemical processing, etc., and based on strengths of similarities and/or differences between the various types of production processing and stages/phases/steps of production processing), for example, and optionally may be delineated further based on other characteristics of the components, if desired. To illustrate, FIG. 4 depicts just one of many different examples of an overall component categorization structure or framework 400 that includes a plurality of component categories and several nested levels of sub-categories.

In some scenarios, the plurality of component categories may include one or more categories of a set of categories including commodity or basic chemicals, semi-complex chemicals, fine chemicals and/or polymers that are derived from petroleum and/or from natural feedstocks (e.g., via biotechnological processes and/or via processes that are not biotechnological), biological materials and/or biological ferments that are directly used, refined, and/or chemically modified; and minerals that are directly used, refined, and/or chemically modified. In some scenarios, the plurality of component categories may include all of the categories of the set of categories including commodity or basic chemicals, semi-complex chemicals, fine chemicals and/or polymers that are derived from petroleum and/or from natural feedstocks (e.g., via biotechnological processes and/or via processes that are not biotechnological), biological materials and/or biological ferments that are directly used, refined, and/or chemically modified; and minerals that are directly used, refined, and/or chemically modified.

Indeed, in some embodiments (not shown FIG. 3), the method 300 may include determining, defining, delineating, or otherwise identifying at least a portion of the plurality of component categories, such as in manners described elsewhere within this document. Generally speaking, the production processes based on which component categories may be delineated or defined may include, for example, the procurement of feedstocks corresponding to the various components used or for use in the ingredients of personal care products, the processing of the procured feedstocks into the components used or for use in the ingredients of personal care products, and the waste disposal and/or waste mitigation performed based on the procurement of the feedstocks and the processing of the procured feedstocks into the components used or for use in the ingredients of personal care products. Accordingly, in embodiments, the determining, defining, delineating, or otherwise identifying the at least a portion of the plurality of component categories may include classifying the chemical structures of components. Further, in some implementations, the determining, defining, delineating, or otherwise identifying the at least a portion of the plurality of component categories may further include classifying feedstock sources of components, and may still further include classifying the production processes of the components into ingredients of personal care products. Of course, the component categories may be delineated or defined further and/or alternately based on other production processes, such as chemical processing of components into ingredients, purifying chemically-processed components, and/or disposing of and/or mitigating at least a portion of the waste generated by the additional production processes.

At a block 308, the method 300 may include, for each candidate component included in the plurality of candidate components, determining a respective e-impact rating of its respective component category. The e-impact rating of a component category may be indicative of an environmental impact of utilizing any one or more of the components of the component category in the ingredients of personal care products. In some implementations, the respective e-impact rating of the component category may be at least partially indicative of an environmental impact of waste generated from utilizing any one or more of the components of the component category in ingredients of personal care products.

The e-impact rating of a component category may be based on an average of individual e-impact ratings of a group of components included in the component category. As such, in some embodiments, the method 300 may include determining the average of the individual e-impact ratings of the group of components included in component category. Typically, but not necessarily, the group of components based on which the average is determined may include a majority of the components included in the component category, and in some cases the group of components may include all of the components included in the component category. In some implementations, the average of the e-impact ratings of the group of components may be a weighted average. Accordingly, in some embodiments, the method 300 may include determining the weighting technique that is utilized to determine the weighted average. The weighting technique may include one or more of the weighting techniques described elsewhere in this document, for example.

Additionally, as the e-impact rating of a component category may be indicative of an environmental impact posed by waste that is generated by utilizing, in ingredients of personal care products, at least some of (e.g., any one or more of) the plurality of components included in the component category, the determining 308 of the respective e-impact rating of the respective component category may include determining a respective amount of environmental hazard posed by the net waste that is generated across multiple different stages or phases (e.g., cradle-to-gate stages or phases) in the production processing of any one or more components of the component category into one or more ingredients of personal care products. Such production processing stages or phases based on which an e-impact rating of a component category may be determined 308 may include, for example, procuring feedstock(s) corresponding to the components included in the category; processing the procured feedstock(s) into the components of the category; chemically processing the components of the category, purifying the chemically-processed components of the category, and/or disposing of and/or mitigating at least a portion of the waste that is respectively generated by each production processing stage or phase contributing to the net waste. In an embodiment, the production processing phases based on which an e-impact rating of a component category may be determined 308 include the procurement of feedstocks corresponding the components of the category, the processing of the procured feedstocks into the components of the category, and the disposing of and/or mitigating at least a portion of the waste generated from the procurement and processing of the feedstocks.

The waste (e.g., the net waste) that is generated by the production processing of any one or more components of the category into ingredients of personal care products may include waste generated from or based on one or more factors related to the various stages or phases of production processing such as, for example, reagents, auxiliaries, catalysts, reaction mechanisms, reaction aids, solvents, reaction byproducts, removed impurities, unreacted starting materials, greenhouse gas emissions, solvent recovery, solvent loss, purification, equipment cleaning processes, manufacturing of starting materials, recycling, upcycling, gasification, composting, utilization of at least a portion of the waste as microbial feedstock, processing through aqueous waste treatment plants, landfilling, byproduct recovery, and/or hazard quality of the waste due to the utilization of the particular candidate component in ingredients of personal care products, to name a few. In embodiments, the waste (e.g., the net waste) that is generated by the production processing of components that are included in the category and/or otherwise utilizing the components that are included in the category in ingredients of personal care products may include waste generated from or based on at least two of said factors. In embodiments, the waste (e.g., the net waste) that is generated by the production processing of and/or otherwise utilizing components of the component category in ingredients of personal care products may include waste generated from or based on a majority or even all of said factors discussed above.

At a block 310, the method 300 may include, for each candidate component included in the plurality of candidate components, assigning the respective e-impact rating of the respective component category of the each candidate component as a respective e-impact rating of the each candidate component. As such, in embodiments, each candidate component included in a particular component category may be assigned a same e-impact rating, e.g., the e-impact rating of the particular component category.

In some embodiments (not shown in FIG. 3), the method 300 may include adjusting or altering the determined respective e-impact rating of each candidate component based on the plurality of candidate components, e.g., in a relative manner with respect to other candidate components. For example, the method 300 may include weighting the initial e-impact rating of each candidate component and/or scaling the initial e-impact rating of each candidate component (e.g., in conjunction with the initial e-impact ratings of other candidate components), for example, in manners similar to those described elsewhere within this disclosure. In an implementation, the adjusted or altered initial component e-impact rating of each candidate component may be indicative of the relativity of the initial e-impact rating of each candidate component with respect to the initial e-impact ratings of other candidate components.

In some embodiments (not shown in FIG. 3), the method 300 further comprises modifying the respective e-impact rating of the each candidate component based on a respective e-hazard adjustment corresponding to the respective component category in which the each candidate component is included, thereby generating a respective enhanced e-impact rating of the each candidate component. For embodiments in which the e-impact ratings of the candidate components are adjusted or altered, the method 300 may include modifying the adjusted or altered respective e-impact rating of each candidate component based on the respective e-hazard adjustment corresponding to the respective component category of the candidate component. As described elsewhere herein, an e-hazard adjustment of a component category may be indicative of an amount of environmental hazard posed by waste that is generated by utilizing, in the ingredients of personal care products, at least some of (e.g., any one or more of) the components included in the component category.

In some embodiments (also not shown in FIG. 3), the method 300 may additionally include integrating the respective e-hazard adjustments with or into a tool that is configured (and executes) to evaluate, assess, and/or measure the environmental impacts of the processing (e.g., production processing) of different components for use in personal care products, and/or the environmental impacts of utilizing the different components themselves in personal care products, where the integrating updates the tool. The tool may be a present-day Green Score tool or a similar, and the present-day Green Score tool (or similar) may be included in the system 100 or may be located remotely with respect to the system 100. In an example implementation, a present-day Green Score tool may be updated to utilize one or more e-hazard adjustments (e.g., as determined by the method 300) so that the updated tool is configured to evaluate, based at least on the integrated e-hazard adjustment(s), environmental impacts of waste generated from the production processing of components for use in personal care products and/or environmental impacts of the utilization of the components themselves sin personal care products. Consequently, scores or measurements generated by the updated tool may be at least partly based on the one or more e-hazard adjustments which were integrated with or into the updated tool.

At a block 312, the method 300 may include selecting, based on a comparison of the enhanced e-impact ratings of the plurality of candidate components (e.g., as stored in the component enhanced e-impact ratings data store 122 and/or as determined in-line with the execution of the method 300), an ingredient that includes a particular component of the plurality of candidate components, where the ingredient may be utilized in a formulation of a personal care product. Generally speaking, the particular component included in the ingredient may have a more environmentally preferred enhanced e-impact rating as compared to other candidate components which may be included in the ingredient. Consequently, the selecting 312 of the ingredient may include selecting an ingredient that is produced from a more environmentally preferred component rather than from other less environmentally preferred candidate components. Additionally or alternatively, the selecting 312 of the ingredient may include selecting an ingredient that is produced from a more environmentally preferred component rather than selecting other ingredients that are processed from less environmentally preferred components. Indeed, in some embodiments (not shown in FIG. 3), the method 300 may further include determining, based on the enhanced e-impact rating of the particular component and optionally based on respective enhanced e-impact ratings of other components, a respective e-impact rating of each ingredient of one or more candidate ingredients which may be included in or otherwise processed into a personal care product (e.g., either alone or in combination with other ingredients), and the selecting 312 of the ingredient may be based on a comparison of the e-impact ratings of the one or more candidate ingredients. Additionally, in some embodiments (not shown in FIG. 3), the method 300 may include utilizing the selected ingredient in which the particular component is included in a formulation of the personal care product and/or in the manufacturing or producing of the personal care product, e.g., based on the formulation.

In some embodiments (not shown in FIG. 3), the method 300 may further include determining, based on the enhanced e-impact rating of the particular component and optionally based on respective enhanced e-impact ratings of other components, a respective e-impact rating of each formulation of one or more candidate formulations which may be utilized to produce or manufacture the personal care product, and/or selecting a particular formulation of the personal care product based on a comparison of the e-impact ratings of the one or more candidate formulations. As such, the selecting of the particular formulation may include selecting a formulation that utilizes a more environmentally preferred component and/or ingredient rather than utilizes other less environmentally preferred components and/or ingredients, and/or the selecting of the particular formulation may include selecting a formulation that is more environmentally preferred as compared to other candidate formulations. The method 300 may additionally include utilizing the selected formulation to manufacture or produce the personal care product.

At a block 315, the method 300 may include providing an indication of the selected ingredient to at least one of a computer application or a user interface to thereby cause the selected ingredient to be utilized in the manufacturing of the personal care product. For example, at the block 315, the indication of the selected ingredient may be provided to one or more local user interfaces 132, and/or the indication of the selected ingredient may be provided via the communication interface(s) 130 to one or more remote user interfaces, one or more other computing devices, one or more other systems utilized in the production of personal care products (such as a formulation system, an automation system, a manufacturing system, a control system, an inventory system, a supply chain management system, etc.), and/or one or more computing applications executing on the other computing devices and/or on the other systems. In some arrangements, such as when the system 100 is integral with another system utilized in the production of personal care products (such as a formulation system, an automation system, a manufacturing system, a control system, an inventory system, a supply chain management system, etc.), at the block 315 the method 300 may include providing the indication of the selected ingredient to a computing application executing on the integral system, e.g., as represented by the engines/instructions 128, and the computing application may execute to generate a formulation and/or to control an industrial plant to produce the personal care product using the formulation, for example.

This disclosure is intended to explain how to fashion and use various embodiments in accordance with the technology rather than to limit the true, intended, and fair scope and spirit thereof. The foregoing description is not intended to be exhaustive or to be limited to the precise forms disclosed. Modifications or variations are possible in light of the above teachings. The embodiment(s) were chosen and described to provide the best illustration of the principle of the described technology and its practical application, and to enable one of ordinary skill in the art to utilize the technology in various embodiments and with various modifications as are suited to the particular use contemplated. All such modifications and variations are within the scope of the embodiments as determined by the appended claims, as may be amended during the pendency of this application for patent, and all equivalents thereof, when interpreted in accordance with the breadth to which they are fairly, legally and equitably entitled.

Moreover, in the foregoing specification and the attached drawings, specific examples have been described. However, one of ordinary skill in the art appreciates that various modifications and changes can be made in view of aspects of this disclosure without departing from the scope of the disclosure as set forth in the claims below. Accordingly, the specification and figures are to be regarded in an illustrative rather than a restrictive sense, and all such modifications made in view of aspects of this disclosure are intended to be included within the scope of present teachings. Numerous alternative examples could be implemented, using either current technology or technology developed after the filing date of this patent, which would still fall within the scope of the claims. By way of example, and not limitation, the disclosure herein contemplates at least the following examples:
1. A method of producing a personal care product with environmentally preferred ingredients, the method comprising:
obtaining a plurality of candidate components for an ingredient of a personal care product;
for each candidate component included in the plurality of candidate components:
determining a respective initial e-impact rating of the each candidate component, the respective e-impact rating of the each candidate component indicative of an environmental impact of utilizing the each candidate component in ingredients of personal care products; determining a respective e-hazard adjustment indicative of a respective amount of environmental hazard posed by waste that is generated by utilizing one or more components, of a respective component category in which the each candidate component is included, in the ingredients of personal care products, the respective component category included in a plurality of categories of components used in the ingredients of personal care products, the plurality of component categories delineated based on chemical structures of the components, feedstock sources of the components, and production processes of the components, and each component category of the plurality of component categories including a respective plurality of components used in the ingredients of personal care products; and modifying the respective initial e-impact rating of the each candidate component based on the respective e-hazard adjustment corresponding to the respective component category, thereby generating a respective enhanced e-impact rating of the each candidate component;
selecting, based on a comparison of the enhanced e-impact ratings of the plurality of candidate components, an ingredient that includes a particular component of the plurality of candidate components; and
providing an indication of the selected ingredient to at least one of a computer application or a user interface to thereby cause the selected ingredient to be utilized in the manufacturing of the personal care product.
2. The method of the preceding example, further comprising utilizing the selected ingredient in which the particular component is included in producing a formulation of the personal care product.
3. The method of any one of the preceding examples, further comprising utilizing the selected ingredient in which the particular component is included in the manufacturing of the personal care product.
4. The method of any one of the preceding examples, wherein the respective e-hazard adjustment corresponding to the respective component category is indicative of the respective amount of environmental hazard posed by the waste that is generated by the utilizing of the one or more components of the respective component category in the ingredients of personal care products relative to amounts of environmental hazards posed by waste that is generated by utilizing components corresponding to other component categories in the ingredients of personal care products.
5. The method of any one of the preceding examples, wherein the waste that is generated by the utilizing of the one or more components of the respective component category in the ingredients of personal care products includes waste generated from processing respective feedstocks into the one or more components of the respective component category.
6. The method of the preceding example, wherein the waste that is generated by the utilizing of the one or more components of the respective component category in the ingredients of personal care products further includes waste generated from procuring the respective feedstocks from corresponding feedstock sources for processing into the one or more components of the respective component category.
7. The method of any one of the preceding examples, wherein the respective amount of environmental hazard posed by the waste that is generated by the utilizing, in the ingredients of personal care products, of the one or more components of the respective component category in which the each candidate component is included is based on at least two of: reagents, auxiliaries, catalysts, reaction mechanisms, reaction aids, solvents, reaction byproducts, removed impurities, unreacted starting materials, greenhouse gas emissions, solvent recovery, solvent loss, purification, equipment cleaning processes, manufacturing of starting materials, recycling, upcycling, gasification, composting, utilization of at least a portion of the waste as microbial feedstock, processing through aqueous waste treatment plants, landfilling, byproduct recovery, or hazard quality of the waste due to the utilization, in the ingredients of personal care products, of the one or more components of the respective component category in which the each candidate component is included.
8. The method of the preceding example, wherein the respective amount of environmental hazard posed by the waste that is generated by the utilizing, in the ingredients of personal care products, of the one or more components of the respective component category in which the each candidate component is included is based on all of: the reagents, the auxiliaries, the catalysts, the reaction mechanisms, the reaction aids, the solvents, the reaction byproducts, the removed impurities, the unreacted starting materials, the greenhouse gas emissions, the solvent recovery, the solvent loss, the purification, the equipment cleaning processes, the manufacturing of starting materials, the recycling, the upcycling, the gasification, the composting, the utilization of at least a portion of the waste as microbial feedstock, the processing through aqueous waste treatment plants, the landfilling, the byproduct recovery, and the hazard quality of the waste due to the utilization, in the ingredients of personal care products, of the respective plurality of the one or more components of the respective component category in which the each candidate component is included.
9. The method of any one of the preceding examples, wherein the production processes of the components used in the ingredients of personal care products include procurement of feedstocks corresponding to the components used in the ingredients of personal care products, processing of the procured feedstocks into the components used in the ingredients of personal care products, and waste disposal and/or waste mitigation caused by the procurement of the feedstocks and/or the processing of the procured feedstocks into the components used in the ingredients of personal care products.
10. The method of the preceding examples, wherein the procurement of the feedstocks includes at least one of: mining, harvesting, refining, agricultural production, mineral processing, chemical synthesis, chemical modification, biological derivation, fermentation, enzymatic catalysis, another type of procurement that utilizes a biological organism or convergent synthesis.
11. The method of any one of examples 9-10, wherein the processing of the procured feedstocks into the components used in the ingredients of personal care products includes purification.
12. The method of any one of examples 9-11, wherein the waste disposal and/or waste mitigation includes at least one of recycling, upcycling, gasification, composting, utilization as microbial feedstock, processing through aqueous waste treatment plants, landfilling, or another waste mitigation technique.
13. The method of any one of the preceding examples, wherein the determining of the respective initial e-impact rating of the each candidate component includes: determining a respective e-impact rating of the respective component category in which the each candidate component is included, the respective e-impact rating of the respective component category indicative of an environmental impact posed by waste that is generated by utilizing any one component of multiple components included in the respective component category in the ingredients of personal care products; and assigning the respective e-impact rating of the respective component category as the respective initial e-impact rating of the each candidate component.
14. The method of the preceding example, wherein the respective e-impact rating of the respective component category is based on an average of e-impact ratings of an entirety of the respective plurality of components included in the respective component category.
15. The method of the preceding example, wherein the average of the e-impact ratings of the entirety of the respectively plurality of components included in the respective component category is a weighted average.
16. The method of any one of the preceding examples, wherein the determining of the respective initial e-impact rating of the each candidate component includes determining a respective amount of environmental hazard posed by waste that is generated by at least one of: procuring feedstocks corresponding to the each candidate component; processing, into the each candidate component, the procured feedstocks corresponding to the each candidate component; or disposing of and/or mitigating the generated waste corresponding to at least one of the procuring of the feedstocks corresponding to the each candidate component or the processing of the procured feedstocks corresponding to the each candidate component.
17. The method of the preceding example, wherein the disposing and/or the mitigating of the generated waste corresponding to the at least one of the procuring of the feedstocks corresponding to the each candidate component or the processing of the procured feedstocks corresponding to the each candidate component includes disposing and/or mitigating greenhouse gas emissions generated from the procuring of the feedstocks corresponding to the each candidate component and from the processing of the procured feedstocks of the each candidate component into the each candidate component.
18. The method of any one of the preceding examples, wherein the determining of the respective initial e-impact rating of the each candidate component includes categorizing the each candidate component into a respective component category included in the plurality of component categories, the categorization of the each candidate component based on a chemical structure of the each candidate component.
19. The method of any one of the preceding examples, wherein the determining of the respective initial e-impact rating of the each candidate component includes categorizing the each candidate component into a respective component category included in the plurality of component categories, the categorization of the each candidate component based one or more feedstock sources of the candidate component.
20. The method of any one of examples 18-19, wherein the categorizing of the each candidate component into the respective component category is further based on one or more production processes of the each candidate component.
21. The method of any one of the preceding examples, wherein: the respective e-hazard adjustment is a penalty, and generating the enhanced initial e-impact rating of the each candidate component based on the respective e-hazard adjustment includes generating a respective enhanced e-impact rating that indicates the each candidate component is less environmentally-preferred than indicated by the respective initial e-impact rating of the each candidate component.
22. The method of any one of the preceding examples, wherein: the respective e-hazard adjustment is a bonus, and generating the enhanced initial e-impact rating of the each candidate component based on the respective e-hazard adjustment includes generating a respective enhanced e-impact rating that indicates the each candidate component is more environmentally-preferred than indicated by the respective initial e-impact rating of the each candidate component.
23. The method of any one of examples 21-22, wherein: the respective e-hazard adjustment is an additive or subtractive adjustment, and modifying the respective initial e-impact rating of the each candidate component based on the respective e-hazard adjustment includes adding or subtracting the respective e-hazard adjustment to or from the respective initial e-impact rating of the each candidate component.
24. The method of any one of examples 21-22, wherein: the respective e-hazard adjustment is a factor or a multiplier, and modifying the respective initial e-impact rating of the each candidate component based on the respective e-hazard adjustment includes multiplying the respective initial e-impact rating of the each candidate component and the respective e-hazard adjustment.
25. The method of any one of the preceding examples, further comprising scaling the respective initial e-impact rating of the each candidate component based on the plurality of candidate components; and wherein modifying the respective initial e-impact rating of the each candidate component includes modifying the respective, scaled initial e-impact rating of the each candidate component.
26. The method of the preceding example, wherein scaling the respective initial e-impact rating of the each candidate component includes scaling the respective initial e-impact rating of the each candidate component via a logarithmic transformation.
27. The method of any one of the preceding examples, further comprising determining at least one of an e-impact rating of the selected ingredient or an e-impact rating of a formulation in which the selected ingredient is included based on the enhanced e-impact rating of the particular component.
28. The method of any one of the preceding examples, further comprising determining an e-impact rating of a formulation of the personal care product based on the enhanced e-impact rating of the particular component.
29. The method of any one of the preceding examples, further comprising: integrating the respective e-hazard adjustment corresponding to the respective component category with a tool configured to evaluate at least one of: environmental impacts of processing at least one component for use in personal care products, or environmental impacts of using the at least one component in personal care products, thereby generating an updated tool; and utilizing the updated tool to thereby provide an indication of a score or measure of at least one of: an environmental impact of processing the particular component that is included in the respective component category for use in a personal care product, or an environmental impact of utilizing the particular component in the personal care product, the score or measure being at least partly based the respective e-hazard adjustment of the respective component category.
30. A system configured to perform the method of any one of examples 1-29.
31. A method of producing a personal care product with environmentally preferred ingredients, the method comprising:
obtaining an indication of a plurality of candidate components for an ingredient of a personal care product;
for each candidate component included in the plurality of candidate components:
determining a respective component category, of a plurality of categories of components used in ingredients of personal care products, in which the each candidate component is included, the plurality of component categories delineated based on chemical structures of the components, feedstock sources of the components, and production processes of the components, and each component category of the plurality of component categories including a respective plurality of components used in the ingredients of personal care products; determining a respective e-impact rating of the respective component category, the respective e-impact rating of the respective component category indicative of an environmental impact posed by waste that is generated by utilizing at least some of the respective plurality of components of the respective component category in the ingredients of personal care products; and assigning the respective e-impact rating of the respective component category as a respective e-impact rating of the each candidate component;
selecting, based on a comparison of the e-impact ratings of the plurality of candidate components, an ingredient that includes a particular candidate component of the plurality of candidate components; and
providing an indication of the selected ingredient to at least one of a computer application or a user interface.
32. The method of the preceding example, further comprising utilizing the selected ingredient in which the particular component is included in producing a formulation of the personal care product.
33. The method of any one of examples 31-32, further comprising utilizing the selected ingredient in which the particular component is included in the manufacturing of the personal care product.
34. The method of any one of examples 31-33, wherein the respective e-impact rating of the respective component category is based on an average of e-impact ratings of an entirety of the respective plurality of components included in the respective component category.
35. The method of the preceding example, wherein the average of the e-impact ratings of the entirety of the respective plurality of components included in the respective component category is a weighted average.
36. The method of any one of examples 31-35, wherein the determining of the respective e-impact rating of the respective component category includes determining a respective amount of environmental hazard posed by waste that is generated by at least one of: procuring feedstocks corresponding to the at least some of the respective plurality of components of the respective component category; processing, into the at least some of the respective plurality of components of the respective component category, the procured feedstocks of the at least some of the respective plurality of components of the respective component category; or disposing of and/or mitigating waste that is generated by at least one of the procuring of the feedstocks corresponding to the at least some of the respective plurality of components of the respective component category or the processing of the procured feedstocks corresponding to the at least some of the respective plurality of components of the respective component category.
37. The method of the preceding example, wherein the disposing of and/or the mitigating of the generated waste corresponding to the at least some of the respective plurality of components of the respective component category includes disposing of and/or mitigating greenhouse gas emissions generated from the procuring of the feedstocks corresponding to the at least some of the respective plurality of components of the respective component category and from the processing of the feedstocks into the at least some of the respective plurality of components of the respective component category.
38. The method of any one of examples 31-37, wherein the production processes of the components used in the ingredients of personal care products include procurement of feedstocks corresponding to the components used in the ingredients of personal care products, processing of the procured feedstocks into the components used in the ingredients of personal care products, and waste disposal and/or waste mitigation caused by the procurement of the feedstocks and the processing of the procured feedstocks into the components used in the ingredients of personal care products.
39. The method of the preceding example, wherein the procurement of the feedstocks includes one or more of: mining, harvesting, refining, agricultural production, mineral processing, chemical synthesis, chemical modification, purification, biological derivation, fermentation, enzymatic catalysis, another type of procurement that utilizes a biological organism, or convergent synthesis.
40. The method of any one of example 31-39, wherein: the respective e-impact rating of the each candidate component is a respective initial e-impact rating of the each candidate component; and the method further comprises modifying the respective initial e-impact rating of the each candidate component based on a respective e-hazard adjustment corresponding to the respective component category in which the each candidate component is included, thereby generating a respective enhanced e-impact rating of the each candidate component, the respective e-hazard adjustment indicative of a respective amount of environmental hazard posed by waste that is generated by utilizing, in the ingredients of personal care products, at least some of the respective plurality of components of the respective component category in which the each candidate component is included.
41. The method of the preceding example, wherein the respective amount of environmental hazard posed by the waste that is generated by utilizing the at least some of the respective plurality of components of the respective component category in which the each candidate component is included is based on two or more of: reagents, auxiliaries, catalysts, reaction mechanisms, reaction aids, solvents, reaction byproducts, removed impurities, unreacted starting materials, greenhouse gas emissions, solvent recovery, solvent loss, purification, equipment cleaning processes, manufacturing of starting materials, recycling, upcycling, gasification, composting, utilization of at least a portion of the waste as microbial feedstock, processing through aqueous waste treatment plants, landfilling, byproduct recovery, or hazard quality of the waste.
42. The method of the preceding example, wherein the respective amount of environmental hazard posed by the waste that is generated by utilizing the at least some of the respective plurality of components of the respective component category in which the each candidate component is included is based on all of: the reagents, the auxiliaries, the catalysts, the reaction mechanisms, the reaction aids, the solvents, the reaction byproducts, the removed impurities, the unreacted starting materials, the greenhouse gas emissions, the solvent recovery, the solvent loss, the purification, the equipment cleaning processes, the manufacturing of starting materials, the recycling, the upcycling, the gasification, the composting, the utilization of at least a portion of the waste as microbial feedstock, the processing through aqueous waste treatment plants, the landfilling, the byproduct recovery, and the hazard quality of the waste.
43. The method of any one of examples 40-42, further comprising: integrating the respective e-hazard adjustment corresponding to the respective component category with a tool configured to evaluate at least one of: environmental impacts of processing at least one component for use in personal care products, or environmental impacts of utilizing the at least one component in personal care products, thereby generating an updated tool; and utilizing the updated tool to thereby provide an indication of a score or measure of at least one of: an environmental impact of processing the particular candidate component for use in the personal care product, or an environmental impact of utilizing the particular candidate component in the personal care product, the score or measure being at least partly based the respective e-hazard adjustment of the respective component category.
44. The method of any one of examples 40-43, further comprising scaling the initial e-impact ratings of the plurality of candidate components; and wherein the modifying of the respective initial e-impact rating of the each candidate component includes modifying the respective scaled, initial e-impact rating of the each candidate component.
45. The method of any one of examples 31-44, wherein the plurality of component categories includes: commodity or basic chemicals, semi-complex chemicals, fine chemicals and/or polymers that are derived from petroleum and/or from natural feedstocks, biological materials and/or biological ferments that are directly used, refined, and/or chemically modified, and minerals that are directly used, refined, and/or chemically modified.
46. The method of any one of examples 31-45, further comprising determining at least one of an e-impact rating of the selected ingredient or an e-impact rating of a formulation in which the selected ingredient is included based on the e-impact rating of the particular component.
47. The method of any one of examples 31-46, further comprising determining an e-impact rating of a formulation of the personal care product based on the e-impact rating of the particular component.
48. The method of any one of examples 31-47, further comprising classifying the chemical structures of the components used in the ingredients of personal care products, thereby determining the plurality of component categories.
49. The method of the preceding example, further comprising classifying the feedstock sources of the components used in the ingredients of personal care products in conjunction with the classifying of the chemical structures of the components to thereby determine the plurality of component categories.
50. The method of the preceding example, further comprising classifying the production processes of the components used in ingredients of personal care products in conjunction with the classifying of the feedstock sources of the components and the classifying of the chemical structures of the components to thereby determine the plurality of component categories.
51. A system configured to perform the method of any one of examples 31-50.
52. A system configured to perform the method of any one of examples 1-29 and the method of any one of examples 31-51.
53. Any one of the preceding examples in combination with any other one of the preceding examples.

Further, although the foregoing text sets forth a detailed description of numerous different embodiments, it should be understood that the scope of the patent is defined by the words of the claims set forth at the end of this patent. The detailed description is to be construed as exemplary only and does not describe every possible embodiment because describing every possible embodiment would be impractical, if not impossible. Numerous alternative embodiments could be implemented, using either current technology or technology developed after the filing date of this patent, which would still fall within the scope of the claims and all equivalents thereof.

What is claimed:

1. A method of producing a personal care product with environmentally preferred ingredients, the method comprising:
obtaining a plurality of candidate components for an ingredient of a personal care product;
for each candidate component included in the plurality of candidate components:
determining a respective initial e-impact rating of the each candidate component, the respective e-impact rating of the each candidate component indicative of an environmental impact of utilizing the each candidate component in ingredients of personal care products;
determining a respective e-hazard adjustment indicative of a respective amount of environmental hazard posed by waste that is generated by utilizing one or more components, of a respective component category in which the each candidate component is included, in the ingredients of personal care products, the respective component category included in a plurality of categories of components used in the ingredients of personal care products, the plurality of component categories delineated based on chemical structures of the components, feedstock sources of the components, and production processes of the components, and each component category of the plurality of component categories including a respective plurality of components used in the ingredients of personal care products; and
modifying the respective initial e-impact rating of the each candidate component based on the respective e-hazard adjustment corresponding to the respective component category, thereby generating a respective enhanced e-impact rating of the each candidate component;
selecting, based on a comparison of the enhanced e-impact ratings of the plurality of candidate components, an ingredient that includes a particular component of the plurality of candidate components; and
providing an indication of the selected ingredient to at least one of a computer application or a user interface to thereby cause the selected ingredient to be utilized in manufacturing the personal care product.

2. The method of claim 1, further comprising utilizing the selected ingredient in which the particular component is included in at least one of: producing a formulation of the personal care product, or the manufacturing of the personal care product.

3. The method of claim 1, wherein the respective e-hazard adjustment corresponding to the respective component category is indicative of the respective amount of environmental hazard posed by the waste that is generated by the utilizing of the one or more components of the respective component category in the ingredients of personal care products relative to amounts of environmental hazards posed by waste that is generated by utilizing components corresponding to other component categories in the ingredients of personal care products.

4. The method of claim 1, wherein the respective amount of environmental hazard posed by the waste that is generated by the utilizing, in the ingredients of personal care products, of the one or more components of the respective component category in which the each candidate component is included is based on at least two of: reagents, auxiliaries, catalysts, reaction mechanisms, reaction aids, solvents, reaction byproducts, removed impurities, unreacted starting materials, greenhouse gas emissions, solvent recovery, solvent loss, purification, equipment cleaning processes, manufacturing of starting materials, recycling, upcycling, gasification, composting, utilization of at least a portion of the waste as microbial feedstock, processing through aqueous waste treatment plants, landfilling, byproduct recovery, or hazard quality of the waste due to the utilization, in the ingredients of personal care products, of the one or more components of the respective component category in which the each candidate component is included.

5. The method of claim 1, wherein the production processes of the components used in the ingredients of personal care products include procurement of feedstocks corresponding to the components used in the ingredients of personal care products, processing of the procured feedstocks into the components used in the ingredients of personal care products, and waste disposal and/or waste mitigation caused by the procurement of the feedstocks and/or the processing of the procured feedstocks into the components used in the ingredients of personal care products.

6. The method of claim 5, wherein at least one of:
the procurement of the feedstocks includes at least one of: mining, harvesting, refining, agricultural production, mineral processing, chemical synthesis, chemical modification, biological derivation, fermentation, enzymatic catalysis, another type of procurement that utilizes a biological organism, or convergent synthesis; or
the waste disposal and/or waste mitigation includes at least one of recycling, upcycling, gasification, composting, utilization as microbial feedstock, processing through aqueous waste treatment plants, landfilling, or another waste mitigation technique.

7. The method of claim 1, wherein the determining of the respective initial e-impact rating of the each candidate component includes:
determining a respective e-impact rating of the respective component category in which the each candidate component is included, the respective e-impact rating of the respective component category indicative of an environmental impact posed by waste that is generated by utilizing any one component of multiple components included in the respective component category in the ingredients of personal care products; and
assigning the respective e-impact rating of the respective component category as the respective initial e-impact rating of the each candidate component.

8. The method of claim 7, wherein the respective e-impact rating of the respective component category is based on an average of e-impact ratings of an entirety of the respective plurality of components included in the respective component category.

9. The method of claim 1, wherein the determining of the respective initial e-impact rating of the each candidate component includes determining a respective amount of environmental hazard posed by waste that is generated by at least one of:
procuring feedstocks corresponding to the each candidate component;
processing, into the each candidate component, the procured feedstocks corresponding to the each candidate component; or
disposing of and/or mitigating the generated waste corresponding to at least one of the procuring of the feedstocks corresponding to the each candidate component or the processing of the procured feedstocks corresponding to the each candidate component.

10. The method of claim 1, further comprising:
integrating the respective e-hazard adjustment corresponding to the respective component category with a tool configured to evaluate at least one of: environmental impacts of processing at least one component for use in personal care products, or environmental impacts of using the at least one component in personal care products, thereby generating an updated tool; and
utilizing the updated tool to thereby provide an indication of a score or measure of at least one of: an environmental impact of processing the particular component that is included in the respective component category for use in the personal care product, or an environmental impact of utilizing the particular component in the personal care product, the score or measure being at least partly based the respective e-hazard adjustment of the respective component category.

11. A system configured to perform the method of claim 1.

12. A method of producing a personal care product with environmentally preferred ingredients, the method comprising:
obtaining an indication of a plurality of candidate components for an ingredient of a personal care product;
for each candidate component included in the plurality of candidate components:
determining a respective component category, of a plurality of categories of components used in ingredients of personal care products, in which the each candidate component is included, the plurality of component categories delineated based on chemical structures of the components, feedstock sources of the components, and production processes of the components, and each component category of the plurality of component categories including a respective plurality of components used in the ingredients of personal care products;
determining a respective e-impact rating of the respective component category, the respective e-impact rating of the respective component category indicative of an environmental impact posed by waste that is generated by utilizing at least some of the respective plurality of components of the respective component category in the ingredients of personal care products; and
assigning the respective e-impact rating of the respective component category as a respective e-impact rating of the each candidate component;
selecting, based on a comparison of the e-impact ratings of the plurality of candidate components, an ingredient that includes a particular candidate component of the plurality of candidate components; and
providing an indication of the selected ingredient to at least one of a computer application or a user interface.

13. The method of claim 12, further comprising at least one of:
utilizing the selected ingredient in which the particular candidate component is included in producing a formulation of the personal care product; or
utilizing the selected ingredient in which the particular candidate component is included in manufacturing the personal care product.

14. The method of claim 12, wherein the determining of the respective e-impact rating of the respective component category includes determining a respective amount of environmental hazard posed by waste that is generated by at least one of:
procuring feedstocks corresponding to the at least some of the respective plurality of components of the respective component category;
processing, into the at least some of the respective plurality of components of the respective component category, the procured feedstocks of the at least some of the respective plurality of components of the respective component category; or
disposing of and/or mitigating waste that is generated by at least one of the procuring of the feedstocks corresponding to the at least some of the respective plurality of components of the respective component category or the processing of the procured feedstocks corresponding to the at least some of the respective plurality of components of the respective component category.

15. The method of claim 14, wherein the procurement of the feedstocks includes one or more of: mining, harvesting, refining, agricultural production, mineral processing, chemical synthesis, chemical modification, purification, biological derivation, fermentation, enzymatic catalysis, another type of procurement that utilizes a biological organism, or convergent synthesis.

16. The method of claim 12, wherein:
the respective e-impact rating of the each candidate component is a respective initial e-impact rating of the each candidate component; and
the method further comprises modifying the respective initial e-impact rating of the each candidate component based on a respective e-hazard adjustment corresponding to the respective component category in which the each candidate component is included, thereby generating a respective enhanced e-impact rating of the each candidate component, the respective e-hazard adjustment indicative of a respective amount of environmental hazard posed by waste that is generated by utilizing, in the ingredients of personal care products, at least some of the respective plurality of components of the respective component category in which the each candidate component is included.

17. The method of claim 16, wherein the respective amount of environmental hazard posed by the waste that is generated by utilizing the at least some of the respective plurality of components of the respective component category in which the each candidate component is included is based on two or more of: reagents, auxiliaries, catalysts, reaction mechanisms, reaction aids, solvents, reaction byproducts, removed impurities, unreacted starting materials, greenhouse gas emissions, solvent recovery, solvent loss, purification, equipment cleaning processes, manufacturing of starting materials, recycling, upcycling, gasification, composting, utilization of at least a portion of the waste as microbial feedstock, processing through aqueous waste treatment plants, landfilling, byproduct recovery, or hazard quality of the waste.

18. The method of claim 16, further comprising:
integrating the respective e-hazard adjustment corresponding to the respective component category with a tool configured to evaluate at least one of: environmental impacts of processing at least one component for use in personal care products, or environmental impacts of utilizing the at least one component in personal care products, thereby generating an updated tool; and
utilizing the updated tool to thereby provide an indication of a score or measure of at least one of: an environmental impact of processing the particular candidate component for use in the personal care product, or an environmental impact of utilizing the particular candidate component in the personal care product, the score or measure being at least partly based the respective e-hazard adjustment of the respective component category.

19. The method of claim 12, wherein the plurality of component categories includes: commodity or basic chemicals, semi-complex chemicals, fine chemicals and/or polymers that are derived from petroleum and/or from natural feedstocks, biological materials and/or biological ferments that are directly used, refined, and/or chemically modified, and minerals that are directly used, refined, and/or chemically modified.

20. A system configured to perform the method of claim 12.
